(19) United States
(12) Patent Application Publication
(10) Pub. No.: US 2023/0219957 A1
(43) Pub. Date: Jul. 13, 2023

(54) SMALL MOLECULE CB002-ANALOGES RESTORE THE P53 PATHWAY AND TARGET S-PHASE CHECKPOINT

(71) Applicant: Brown University, Providence, RI (US)

(72) Inventors: WAFIK S. EL-DEIRY, PROVIDENCE, RI (US); LIZ J. HERNANDEZ BORRERO, PROVIDENCE, RI (US)

(21) Appl. No.: 17/996,493

(22) PCT Filed: May 17, 2021
(86) PCT No.: PCT/US2021/032770

§ 371 Date: Oct. 18, 2022

Related U.S. Application Data
(60) Provisional application No. 63/025,732, filed on May 15, 2020.

(51) Int. Cl. C07D 473/08; A61P 35/00

(52) U.S. Cl. CPC C07D 473/08; A61P 35/00

(57) ABSTRACT

The inventors had earlier discovered a class of anticancer xanthine-related drugs that restores p53 activity in tumors with mutated p53 and perturbs an S-phase checkpoint. Based upon the properties of this class of p53-pathway restoring drugs, the inventors developed uses in cancer treatment. The invention provides drug combinations as new therapy regimens due to synthetic lethality in cancer models.

### REFERENCE TO RELATED APPLICATIONS

This invention claims priority to provisional patent application U.S. 63/025,732, filed May 15, 2020, titled “Small molecule CB002-analogs restore the p53 pathway and target S-phase checkpoint,” which is incorporated herein by reference in its entirety.

### STATEMENT REGARDING FEDERALLY SPONSORED RESEARCH OR DEVELOPMENT

This invention was made with government support under grant number CA176289 awarded by the National Institutes of Health. The government has certain rights in the invention.

### FIELD OF THE INVENTION

This invention generally relates to heterocyclic compounds containing purine ring systems with oxygen, sulfur, or nitrogen atoms directly attached in positions 2 and 6, two oxygen atoms with radicals containing only hydrogen and carbon atoms, attached in position 1 or 3 with methyl radicals in positions 1 and 3, e.g., theophylline.

### BACKGROUND OF THE INVENTION

About 50% of human cancers have mutations in p53 genes. The other 50% of human cancers involve a type of inactivation within the p53-pathway. To date, there are no FDA-approved drugs for the restoration of the p53-pathway. Unlike other tumor suppressors, p53 mutations result in a stable p53 protein that acquires a gain-of-function activity leading to an aggressive tumor with more metastatic potential and resistance to chemotherapy.

Direct activation of the p53 transcription factor has been challenging. Existing drugs do involve the restoration of the mutant to wild-type p53 conformation, such as HSP90 inhibitors that target the mutant p53 protein for degradation and MDM2 inhibitors that facilitate wild-type p53 activation. However, these drugs are limited to the type of p53 mutation.

The patent application U.S. Pat. Pub. 2019/0225613 A1 (El-Deiry et al.) described a different approach, the screening for small molecules that restore the p53-pathway instead of direct targeting of p53. The '613 application described compounds, compositions, and methods to treat cancer by restoring the P53 pathway signaling to repress cancer cell growth. The '613 application characterized a new class of small molecules that restore the p53.

As of now, there are no US Food & Drug Administration-approved drugs that restore the p53-pathway in any tumors. The 7% five-year survival rate for pancreatic cancer patients remained unchanged for decades. There remains a need in the medical art for different therapeutic interventions.

### SUMMARY OF THE INVENTION

The inventors earlier discovered a class of anticancer xanthine-related drugs that restores p53 activity in tumors with mutated p53 and perturbs an S-phase checkpoint. The inventors developed uses in cancer treatment based upon the properties of this class of p53-pathway restoring drugs. This invention provides beneficial drug combinations as new therapy regimens due to synthetic lethality in cancer models.

In a first embodiment, the invention provides an identified class of CB002-analog xanthine derivatives, having the following chemical structure.

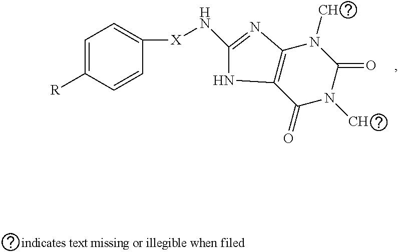

where R=carbon-based substitutions, aromatic groups, halides, aliphatic chains including attached charged groups, molecule dimers, —Cl, or —CF₃; and X=0, 1, more —CH₂, or other linker groups.

The members of this new class of small molecules share a common chemical structure because they contain phenyl groups that differ in their R substituents attached to the purine (xanthine) moiety. The features of this class of CB002-analog xanthine derivatives are: (1) a sub-class of xanthine analogs with very different and unexpected properties as compared to caffeine, pentoxifylline, and theophylline; (2) that are potent in their cytotoxic effects as compared to other known xanthines; (3) that rely on the protein Noxa and autophagy for cell death, which features are not found in caffeine, pentoxifylline, theophylline or other xanthines; (4) by perturbing the S-phase, unlike caffeine, pentoxifylline, theophylline or other xanthines that deregulate the G2-checkpoint. The combination of p53-pathway restoration and S-phase perturbation in this defined class was not predicted by known xanthines like caffeine, pentoxifylline, or theophylline. The class of CB002-analog xanthine molecules is useful as anti-tumor agents with a mechanism of action involving the restoration of p53 pathway signaling and activation of an S-phase checkpoint.

In a second embodiment, the class of compounds is the identified class of CB002-analog xanthine derivatives having the structure described above, with the proviso that the class does not include the compound CB002 or the compound CB002-analog #11.

In a third embodiment, the invention provides CB002-analog #4 (T4). CB002-analog #4 has the following chemical structure:

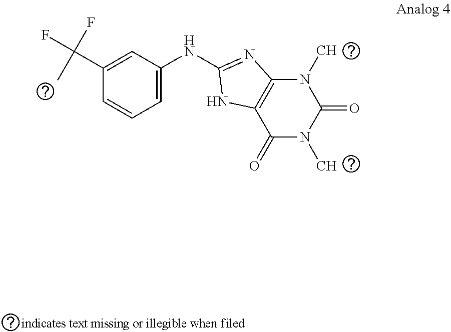 [SMILES: Cn1c(=O)c2[nH]c(Nc3cccc(C(C)(F)F)c3)nc2n(C)c1=O]

R=—CF₃, X=0 —CH₂ CB002-analog #4 can restore the p53-pathway in cancer cells. CB002-analog #4 decreases Ki-67 staining in patient-derived tumor organoids. CB002-analog #4 treatment results in S-phase accumulation notably observed at eight hours following release from synchronization. CB002-analog #4 increases by 30% cells in S-phase at twelve hours, compared to DMSO vehicle control. No significant differences are observed in G2-phase cells between etoposide and CB002-analog #4 at twenty-four hours. S-phase delays with CB002 and CB002-analog #10 occur at six-eight hours of treatment, particularly a 2-fold difference combined with etoposide. This drug is not toxic to normal human fibroblasts. Preliminary in vivo studies demonstrate a lack of toxicity in mice.

In a fourth embodiment, the invention provides CB002-analog #10. CB002-analog #10 has the following chemical structure:

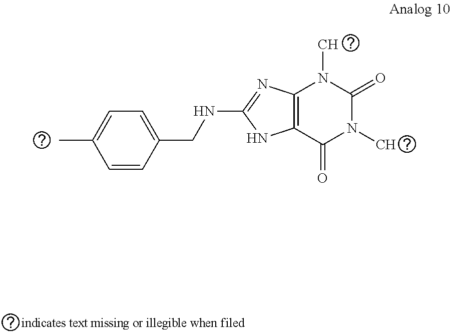

R=—Cl, X=1 —CH₂. CB002-analog #10 treatment results in S-phase accumulation notably observed at 8 hours following release from synchronization. S-phase delays with CB002-analog #10 occur at 6-8 hours of treatment, particularly a 2-fold difference combined with etoposide.

In a fifth embodiment, the invention provides a therapeutic strategy to treat cancer patients. A CB002-analog xanthine compound is administered to a subject or patient who has cancer. The properties of these CB002-analog xanthine compounds differ from other xanthines such as caffeine, pentoxifylline, and theophylline that do not restore p53 pathway signaling in tumors with mutant p53 and which deregulate a G2-checkpoint rather than induce an S-phase checkpoint. Cb002-analogs induce pro-apoptotic NOXA protein, whereas caffeine, pentoxifylline, and theophylline do not. The effectiveness of the therapeutic administration for treating cancer can be determined by any of the cancer diagnostic methods know to persons having ordinary skill in the medical arts.

In a fourth embodiment, the invention provides a therapeutic strategy to treat patients with mutant p53-expressing chemotherapy-resistant colorectal or other solid tumors. Cell cycle analysis of synchronized tumor cells treated with etoposide shows CB002-analogs perturb S-phase, increased p-RPA/RPA2, p-ATR, cyclin E, and decrease p-histone H3 protein expression. Microarray data show that CB002-analogs downregulate essential proteins of the DNA synthesis and repair machinery.

The inventors describe a class CB002-analog xanthine molecules as anti-tumor agents with a unique mechanism of action involving the restoration of p53 pathway signaling and activation of an S-phase checkpoint. The properties of these CB002-analog xanthine compounds differ from other xanthines such as caffeine, pentoxifylline, and theophylline that do not restore p53 pathway signaling in tumors with mutant p53 and which deregulate a G2-checkpoint rather than induce an S-phase checkpoint.

### BRIEF DESCRIPTION OF THE DRAWINGS

For illustration, some embodiments of the invention are shown in the drawings described below. The invention is not limited to the precise arrangements, dimensions, and instruments shown.

FIG. 1 is a pair of graphs showing that CB002 and structural analogs restore the p53 pathway, unlike xanthines caffeine, pentoxifylline, and theophylline. CB002 structural analogs activate p53 reporter gene activity in SW480 cells in a dose-dependent manner (six hours). FIG. 1(A) is a graph showing the therapeutic indices for CB002-structural analogs as determined in SW480 cells (forty-eight hours). FIG. 1(B) is a line graph showing the results of propidium iodide cell cycle analysis performed to determine Sub-G1 population at 48 hours of treatment with CB002 analogs at 100 μM in SW480 cells. Two-way ANOVA, p<0.05. FIG. 1(B) is a bar graph showing the propidium iodide cell cycle analysis performed to determine the Sub-G1 population at forty-eight hours of treatment with CB002 analogs. The statistical analysis was two-way ANOVA, p<0.05. CB002-analog #4 restores the p53 pathway in SW480 cells, resulting in PARP cleavage independently of p73 Immunofluorescence staining was performed using Cyt-C, Tom20, and DAPI in SW480 cells treated for forty-eight hours. CB002 analogs induced Noxa protein expression in DLD-1 cells and SW480 (24 hours). Assays showed that p53-pathway restoring compounds have unique properties compared to other xanthine derivatives in their ability to induce Noxa expression after 24-hour treatment in DLD-1 cells, using caffeine, pentoxifylline, and theophylline. Other assays showed immunofluorescence staining of Cyt-C, Tom20, and DAPI in SW480 treated for forty-eight hours. Noxa protein expression induced by CB002 analogs in DLD-1, SW480, HCT116 and HCT116 p53^{(R175H)} colorectal cancer cells (24 hours). p53-pathway restoring compounds have unique properties compared to other xanthine derivatives in their ability to induce Noxa expression, 24-hour treatment in DLD-1 cells (G). Xanthine derivatives CB002 and its analogs induce Noxa expression but not caffeine, pentoxifylline and theophylline at 24 hours in DLD-1 and SW480 cells.

FIG. 2 is a set of bar graphs showing a reactome pathway analysis of CB002-analog #4. FIG. 2(A) reveals differentially expressed genes in SW480 cells. The upper and lower bar diagrams show enriched pathways corresponding to the T4 (CB002-analog #4 treatment) responsive upregulated and down-regulated genes. The figure analyzes the data collected from the transcriptomic analysis of control (dimethyl sulfoxide, DMSO) versus CB002-analog #4 (T4)-treated samples for twelve hours. FIG. 2(B) provides a reactome pathway analysis of CB002 responsive differentially expressed proteins in SW480 cells. The upper and lower bar diagrams show the enriched pathways corresponding to the CB002 responsive upregulated and downregulated proteins compared with the dimethyl sulfoxide (DMSO) control. This proteomic pathway analysis shows CB002-analog #4 responsive differentially expressed proteins in SW480 cells. Enriched pathways corresponding to the CB002-analog #4 responsive up-regulated (upper) and down-regulated (lower) proteins (in comparison with the DMSO). A heatmap of these results showed the grouped proteins' expression value of some target pathway proteins highlighted in the box area. The figure analyzes data collected from the proteomic analysis of DMSO versus CB002 and analog #4 treated SW480 cell samples for twenty-four hours.

FIG. 3 is a set of graphs showing that CB002-analog #4 is more potent than CB0002 in vitro and in vivo. HCT116 isogenic panel treated with CB002 or analog #4 for forty-eight hours and their respective IC₅₀ values in TABLE 1. FIG. 3(A) is a set of line graphs and FIG. 3(B) is a bar graph, which both show that CB002-analog #4 increases apoptotic cells as shown by the Sub-G1 content in cancer cells but not in normal WI38 cells (forty-eight hours). The statistical analysis was two-way ANOVA, p<0.0001. FIG. 3(C) is a bar graph showing that the 72-hour treatment with CB002-analog #4 is most potent. CB002-analog #4 increases dead cells as shown by the ethidium homodimer staining compared to calcein-stained live cells and cleaved caspase-3 immunofluorescence in colorectal cancer patient-derived organoid cells. CB002-analog #4 decreased ki67 staining in a dose-dependent manner (seventy-two hours) in colorectal cancer patient-derived organoid cells. CB002-analog #4 is non-toxic in vivo (F) and reduces tumor volume in NSG mouse xenografts with SW480 wild-type cells (G) but not in SW480 cells with shNoxa (H). 50 mg/kg by oral gavage three times per week. The statistical analysis was an unpaired t-test, p<0.05.

TABLE 1

| | HCT116 | HCT116 | HCT116 | HCT116 |
|---|---|---|---|---|
| | p53−/− | p53+/+ | p53 R175H | p53 R273H |
| Compound | μM | μM | μM | μM |
| CB002 | 160.9 (95% | 241.7 (95% | 400 | 140 (95% |
| | Cl 114.9 to | Cl 168.1 to | (too wide) | Cl 108.4 to |
| | 280.7) | 482.4) | | 207.9) |
| Analog | 8.831 (95% | 8.691 (95% | 4.462 (95% | 8.091 (95% |
| #4 | Cl 6.625 to | C1 6.659 to | Cl 2.773 to | Cl 5.738 to |
| | 11.79) | 11.35) | 7.123) | 11.41) |

FIG. 4 is a set of chemical drawings showing the parental compound CB002 and its structural analogs. CB002 and its structural analogs are xanthine derivatives that share a common chemical structure that consists of phenyl groups that differ in their R substituents attached to the purine (xanthine) moiety.

FIG. 5 is a scatter plot and a set of volcano-plot charts showing the results of comparative label-free quantitative proteomic analysis of SW480 cell lines in response to dimethyl sulfoxide (DMSO), CB002 (CB), and CB002-analog #4 (T4) treated for twenty-four hours. FIG. 5(A) shows the results of a principal component analysis (PCA) of total protein abundance data collected from dimethyl sulfoxide, CB002 (CB), and CB002-analog #4 (T4) samples. The data represent the close clustering of protein abundance of each replicates under the same group, with variability among the treatments. FIG. 5(B)-(D) shows volcano-plots of fold-change versus p-value of the total of 3743 proteins quantified from SW480 cell lines in response to dimethyl sulfoxide, CB002, and T4 treatments. Red and green circles represent the significant (p<0.05) up and downregulated proteins. Gray circles (p=0.05) are non-significant and below the threshold of fold expression. The inventors performed a heat map and clustering analysis of the total proteins (3743) identified from DMSO, CB and T4 samples.

FIG. 6. is a pair of Venn diagrams showing a transcriptome data comparison with an in-house p53 proteomic database and known p53 targets. CB002-analog #4 (T4) responsive differentially expressed genes in SW480 cells overlap with the in-house p53-proteomic database and with known p53 targets, as described in Fischer, Oncogene, 36(28), 3943-3956 (Jul. 13, 2017). The left and right Venn diagrams show the upregulated and down-regulated CB002-analog #4 (T4) responsive genes overlap with the two known datasets, respectively.

FIG. 7 is a set of four Venn diagrams showing proteomics data comparison CB002-analog #4 (T4) responsive proteins, compared with dimethyl sulfoxide (DMSO) and CB002 comparison with in-house p53-proteomic database and known p53 targets. The left and right Venn diagrams show the upregulated and down-regulated CB002-analog #4 (T4) responsive genes overlap with the two known datasets, respectively. FIG. 7(A) is a three-way Venn diagram of up-regulated analog #4 responsive proteins. FIG. 7(B) is a three-way Venn diagram of down-regulated analog #4 responsive proteins. The data were collected from the proteomic analysis of DMSO versus analog #4 treated SW480 cell samples for 24 hours.

FIG. 8 is a pair of Venn diagrams showing proteomic data comparison, showing proteins increased or decreased in abundance with CB002-analog #4 (T4) treatment compared to DMSO and CB002. The top and bottom Venn diagrams show the upregulated and down-regulated CB002-analog #4 responsive genes overlap with the two known datasets. The two-way Venn diagrams show the up-regulated (top) and down-regulated (down) analog #4 responsive proteins compared to CB002. These data collected from the proteomic analysis of DMSO versus CB002 and analog #4 treated SW480 cell samples for twenty-four hours.

FIG. 9 is a pair of Venn diagrams showing CB002-analog #4 (T4) responsive proteins/genes (DMSO vs. T4), making a comparison of proteomic vs. transcriptome data set. The left and right Venn diagrams show the upregulated and down-regulated CB002-analog #4 (T4) responsive genes overlap with the two known datasets, respectively.

FIG. 10 is a set of Venn diagrams showing transcriptomic pathway analysis of analog #4 reveals differentially expressed genes in tumor cells with mutant p53. SW480 cells were treated with analog #4 for twelve hours. FIG. 2(A) is a three-way Venn diagram of all genes tested that met the low expression cutoff (pink), differentially expressed genes with an FDR<0.05 (purple), and the known p53 target gene set (A). The inventors performed a heatmap analysis of differentially expressed genes that overlapped with the known p53 target gene set. Predictive transcription factor analysis according to direct binding motif was performed for all the differentially expressed genes (total genes 3,362), as shown in TABLE 2. FIG. 10(B) is a four-way Venn diagram of differentially expressed genes with an FDR<0.05 (purple), and the known p53 target gene set from TABLE S3 of Fischer, Oncogene, 36(28), 3943-3956 (2017) (green), ATF4 gene set (yellow) and E2F gene set (pink).

TABLE 2

| Motif | NES | TF HighConf |
|---|---|---|
| dbcorrdb_E2F4_ENCSR000DY Y_1_m1 | 6.36 | E2F4 |
| cisbp_M3134 | 6.25 | E2F1 |
| transfac_public_M00516 | 6.14 | E2F1 |
| dbcorrdb_E2F4_ENCSR000EVL_1_m1 | 5.93 | E2F4 |
| ransfac_pro_M00920 | 5.9 | E2F1; E2F3; E2F4; E2F7 |

FIG. 11 is a set of graphs showing transcriptomic analysis quality control principal component (PC) plots and false discovery rate (FDR) bar graph. PC1 accounts for the highest variability factor being the differences between control and analog #4 treatment. Statistically significant changes in gene expression were determined as FDR<0.05.

### DETAILED DESCRIPTION OF THE INVENTION

### Industrial Applicability

The members of this class of small molecules are therapeutically useful in treating several known cancers.

Colorectal cancer (CRC) is a significant health problem globally, especially in high-risk cohorts with colonic polyps.

Prostate cancer (PC) is the most frequently diagnosed cancer among men in the United States. Prostate cancer is the third cause of cancer mortality. Despite advances in the treatment and understanding of pathogenesis, patients with metastatic Prostate cancers invariably progress to a lethal stage of castration-resistant prostate cancer (CRPC). Inhibition of androgen signaling remains crucial to treat castration-resistant prostate cancer, but treatment strategies are urgently needed.

According to the American Cancer Society, about 10-15 percent of all lung cancer diagnoses are small cell lung cancer (SCLC). The median survival rate of limited-stage small cell lung cancer is about twelve to sixteen months. Several clinically approved chemotherapy drugs were used to treat small cell lung cancer, with several more under clinical trials. However, a combination is still needed to increase patient life expectancy.

Castration-resistant prostate cancer (CRPC) represents a lethal stage of disease with limited treatment options beyond the androgen receptor (AR) inhibitors and chemotherapy.

Brain tumors are one of the most lethal cancers in children and young adults.

Pancreatic ductal adenocarcinoma (PDAC) is one of the deadliest human cancers and still represents an unmet clinical need. Survival, proliferation, and drug resistance of pancreatic cancer cells are driven by KRAS mutation, which supports both mitochondrial and glycolytic bioenergetics.

### Introduction to the Background of the Problem Presented

Tumor suppressor p53 responds to cell stress signals from DNA damage, oncogene activation, oxidative stress and hypoxia. Upon activation by post-translational modifications and oligomerization, p53 signals cell cycle arrest, apoptosis, or DNA repair, according to the extent of the cellular stress, thereby controlling cell fate and preventing tumorigenesis. Riley et al., Nature Rev. Mol. Cell. Biol., 9(5), 402-12 (2008).

Mutations in TP53 commonly occur in most human tumors and confer aggressive tumor phenotypes including metastasis and therapy resistance. TP53 is the most commonly mutated gene (TCGA, 2020), including in ovarian, colorectal, esophageal, head & neck, lung and pancreatic cancers that are the most affected sporadic human cancer types. Olivier, Hollstein, & Hainaut, TP53 mutations in human cancers: origins, consequences, and clinical use. Cold Spring Harb. Perspect. Biol., 2(1), a001008 (2010). TP53 is mutated in over 50% of human cancers and the other 50% involves a biological inactivation of its signaling pathway. The mutated p53 protein results in loss-of-function but due to oligomerization can act in a dominant-negative fashion regarding remaining wild-type p53 allele. Unlike other tumor suppressors, mutant p53 protein can also acquire a gain-of-function which contributes to aggressive tumor phenotypes including enhanced invasion, genomic instability and therapy resistance. Patients whose tumors carry p53 mutations have a poor prognosis and decreased overall survival. Wattel et al., p53 mutations are associated with resistance to chemotherapy and short survival in hematologic malignancies. Blood, 84(9), 3148-57 (1994).

A common feature of cancer cells is genomic instability due to ineffective cell cycle checkpoint responses. Genomic instability is not necessarily due to defective checkpoints, because the checkpoints may be intact but the repair may be deficient. After DNA damage, a healthy cell cycle checkpoint response is to arrest the cell at the G1 phase. Most cancer cells have an ineffective G1-checkpoint due to p53 mutation but retain a functional G2-checkpoint. Most cancer cells can undergo cell arrest at the G2 phase. Cancer cells depend on bypassing intra-S-phase and G2/M checkpoints for unrestrained cell proliferation. Stress signal transduction in the p53 pathway is begun by activating the kinases ataxia-telangiectasia-mutated (ATM), ataxia telangiectasia, and Rad3-related (ATR) and downstream checkpoint kinases Chk1/2 signaling sensors and mediators of p53 activation. It was a long-standing dogma that ATM/Chk2 and ATR/Chk1 are independently activated but recent studies provide evidence of cross-talk between the kinases. Chk1/2 are kinases that participate in cell cycle checkpoint control, with Chk1 being active in S-phase and G2-phase whereas Chk2 is active throughout the cell cycle.

Accumulation of genomic aberrations over time, renders cancer cells vulnerable to checkpoint targeting therapy.

Since the discovery of checkpoint targets, small molecule inhibitors were pursued combined with ionizing radiation and chemotherapy agents to deregulate checkpoints, leading to cancer cell death. A combination of caffeine, a xanthine derivative, with irradiation or chemotherapy agents deregulated the G2-checkpoint through ATM/ATR inhibition leading to therapy sensitization and enhanced cell death. Russell et al., Cancer Drug Design and Discovery (Elsevier/Academic Press, 2008), pages 427-431; and Sarkaria et al., Cancer Res. 59(17), 4375-82 (Sep. 1, 1999). But translational cancer therapeutics studies were discontinued due to unachievable active concentrations in human plasma. More recently, the field focused on developing Chk1/2 inhibitors, which are in clinical trials. Fracasso et al., Cancer Chemother. Pharmacol., 67, 1225-1237 (2011); Huang et al., ACS Med Chem Lett. 3(2), 123-128 (Feb. 9, 2012); and Rogers et al., Cancer Res. (Mar. 11, 2020).

Another cancer therapeutic approach the inventors pursued involves restoration of p53 pathway signaling in tumors with mutant p53 or tumors null for p53. The inventors previously reported a p53-pathway restoring compound CB002 whose mechanism of action was not elucidated. The inventors showed that CB002 leads to apoptotic cell death mediated by p53 target Noxa, a pro-apoptotic protein. Hernandez-Borrero et al., Cell Cycle, 17(5), 557-567 (2018).

Compositions of matter. The inventors have now evaluated more potent CB002-analog compounds and uncovered a unique mechanism of action of a class of anti-cancer drugs. Based on their molecular structure as xanthine derivatives, this class of CB002-analogs, unlike caffeine and other established xanthine derivatives, do not deregulate the G2-checkpoint. These CB002-analog xanthines perturb S-phase. More importantly they restore the p53-pathway, a property not found with caffeine, pentoxifylline and theophylline. The inventors further characterized and defined by transcriptomic and proteomic analysis this new class of small molecules with anti-tumor properties.

These CB002-analogs induce pro-apoptotic Noxa protein in an ATF3/4-dependent manner, whereas caffeine, pentoxifylline, and theophylline do not. By contrast to caffeine, CB002-analogs target an S-phase checkpoint associated with increased p-RPA/RPA2, p-ATR, decreased Cyclin A, p-histone H3 expression and downregulation of essential proteins in DNA-synthesis and DNA-repair. CB002-analog #4 enhances cell death and decreases Ki-67 in patient-derived tumor-organoids without toxicity to normal human cells.

### Compositions of Matter

Substituents of compounds may be disclosed in groups or ranges. The disclosure includes every individual subcombination of the members of such groups and ranges.

For compounds in which a variable appears more than once, each variable can be a different moiety chosen from the Markush group providing options for the variable. For example, where a structure is described as having two R groups simultaneously present on the same compound, the two R groups can represent different moieties chosen from the Markush group defined for R. In another example, when an optionally multiple substituent R is designated in the form,

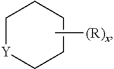

then substituent R can occur x number of times on the ring at any position. R can be a different moiety at each occurrence. Further, in the above example, where the variable Y usually would include one or more hydrogens, such as when Y is CH₂, NH, etc., any H can be replaced with a substituent.

Particular features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable subcombination.

Stereoisomers (including diastereomers and enantiomers) of the compounds described herein, and mixtures thereof, are within the scope of the present disclosure. By way of non-limiting example, the mixture may be a racemate, or the mixture may comprise unequal proportions of one particular stereoisomer over the other. The compounds can be provided as substantially pure stereoisomers. Diastereomers include, for example, cis-trans isomers, E-Z isomers, conformers, and rotamers. Methods of preparation of stereoisomers are known in the medical chemical art, such as by resolving racemic mixtures or by stereoselective synthesis. Many geometric isomers of olefins, C═N double bonds can also be present in the compounds. All such stable isomers are contemplated in this disclosure. Cis and trans geometric isomers of the compounds are also included within the scope of the disclosure. They can be isolated as a mixture of isomers or as separated isomeric forms. Where a compound capable of stereoisomerism or geometric isomerism is designated in its structure or name without reference to specific R/S or cis/trans configurations, all such isomers are contemplated.

Resolution of racemic mixtures of compounds can be carried out by any of numerous methods known in the medical chemical art, including fractional recrystallization using a chiral resolving acid, which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and various optically active camphorsulfonic acids such as β-camphorsulfonic acid. Other resolving agents suitable for fractional crystallization methods include, but are not limited to, stereoisomerically pure forms of a-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent, e.g., dinitrobenzoylphenylglycine. Suitable elution solvent compositions can be determined by one skilled in the medical chemical art.

Appropriate compounds may also include tautomeric forms. Tautomeric forms result from a single bond's swapping with an adjacent double bond with the concomitant migration of a proton. Tautomeric forms include prototropic tautomers, which are isomeric protonation states having the same empirical formula and total charge. Tautomeric forms can be in equilibrium or sterically locked into one form by appropriate substitution.

The compounds also include hydrates and solvates, and anhydrous and non-solvated forms.

The compounds can also include all isotopes of atoms occurring in the intermediates or final compounds. Isotopes include those atoms having the same atomic number but different mass numbers. For example, isotopes of hydrogen include tritium and deuterium. Carbon (¹²C) can be replaced at any position with ¹³C or ¹⁴C. Nitrogen (¹⁴N) can be replaced with ¹⁵N. Oxygen (¹⁶O) can be replaced at any position with ¹⁷O or ¹⁸O. Sulfur (³²S) can be replaced with ³³S, ³⁴S, or ³⁶S. Chlorine (³⁵Cl) can be replaced with ³⁷Cl. Bromine (⁷⁹Br) can be replaced with ⁸¹Br.

In some embodiments, the compounds, or salts thereof, are substantially isolated. Partial separation can include, for example, a composition enriched in any one or more compounds described herein. Methods for isolating compounds and their salts are routine in the medical chemical art.

Compounds containing an amine function can also form N-oxides. A reference herein to a compound that contains an amine function also includes the N-oxide. Where a compound provides several amine functions, one or more than one nitrogen atom can be oxidized to form an N-oxide. Examples of N-oxides include N-oxides of a tertiary amine or a nitrogen atom of a nitrogen-containing heterocycle.

The inventors describe a class of anti-tumor agents with a unique mechanism of action involving restoration of p53 pathway signaling, independently of p53, in tumors with mutated-p53 and characteristics of an S-phase checkpoint. The defining members of this class that best exemplify the mechanistic properties are CB002-analogs #4 and #10. The properties of these CB002-analog xanthine compounds differ other xanthines such as caffeine, pentoxifylline, and theophylline that do not restore p53 pathway signaling in tumors with mutant p53 and which deregulate a G2-checkpoint rather than induce an S-phase checkpoint.

This approach to discovering p53 pathway restoring compounds involved cell-based screening for functional restoration of p53-regulated reporter activity, coupled with cell death induction. Thus, small molecule lead compounds and structural-analogs were not expected to act directly on mutant p53 or restore binding of mutant p53 to genes normally regulated by p53. With the compounds described here, activation of p53 target genes such as Noxa or DR5 occurred independently of p53 and this was observed in tumor cells with different p53 mutations. Thus, there is no expectation that CB002 or analogs #4 or #10 will cause mutant p53 to bind to DNA or chromatin in the regulatory regions of Noxa or DR5 in a manner that wild-type p53 does. The induction of p53 targets occurred independently of p53 family member p73, but in a manner that requires integrated stress response transcription factor proteins ATF3/4. These results provide a molecular mechanism for activation of p53 target genes in a manner that substitutes transcription factors such as ATF3/4 for defective p53. This mechanism results in tumor suppression through induction of pro-apoptotic factors despite p53 mutation, and therefore acts as a bypass mechanism to prevent tumor growth in drug-treated cells.

Use of the compositions of matter for treating cancer. Preliminary in vivo studies demonstrate anti-tumor efficacy in mice. Thus, this class of anti-cancer drugs show activation of p53 pathway signaling in tumors with mutated p53 and target an S-phase checkpoint.

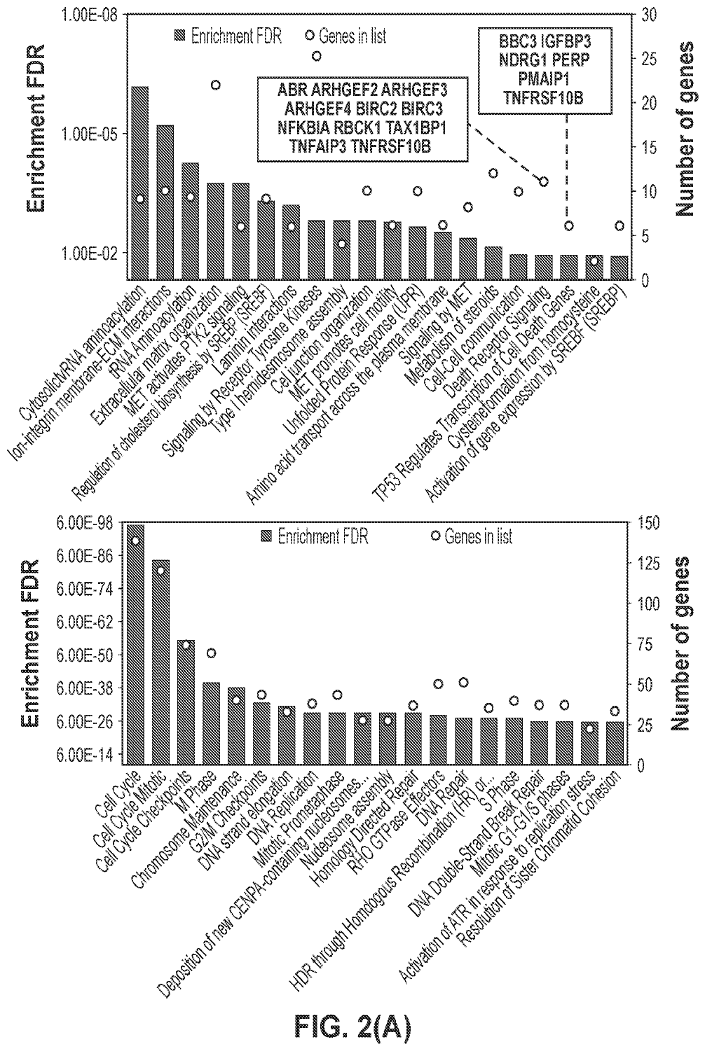

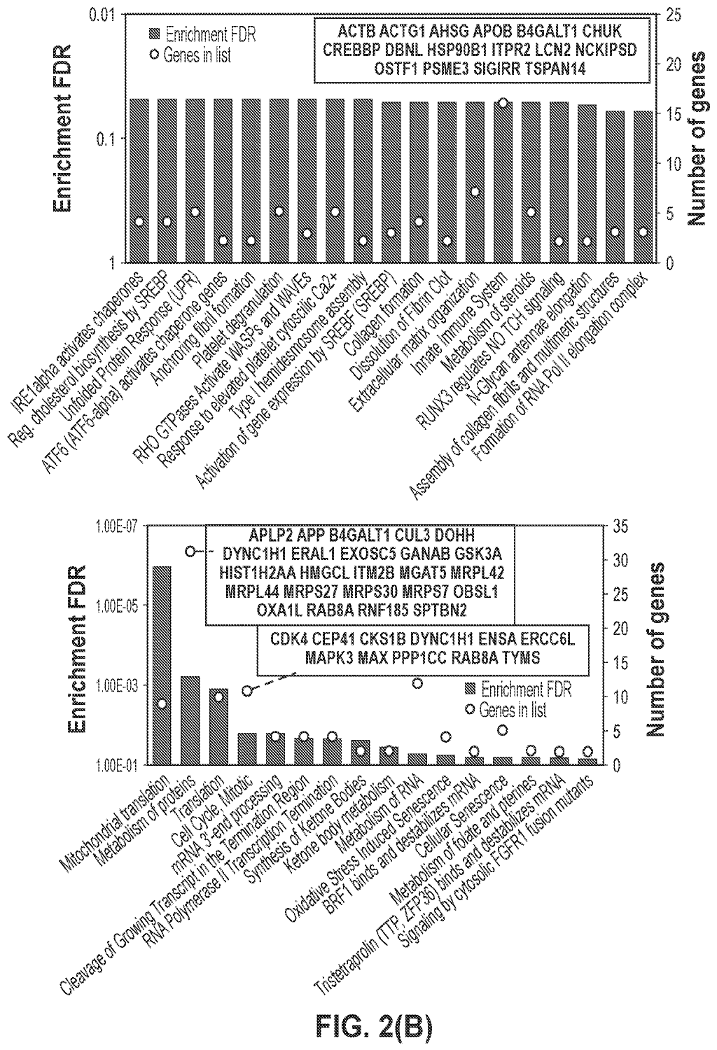

CB002-analog #4 is 20-30 times more potent and like the CB002 parental-compound restores the p53-pathway and induces apoptosis independently of p73. The twelve p53 pathway restoring structural analogs of CB002 tested were similar because they resemble the structure of a xanthine. This transcriptional analysis identified 102 genes involved in the p53-pathway and IPA determined p53 to be activated as an upstream regulator with a z-score value of 3.3 and p-value of 2.9×10⁻³⁴. These data further validates the anti-cancer class of small molecules as p53-restoring drugs. Microarray analysis identified approximately 150 genes involved in cell cycle regulation, DNA synthesis and repair that are decreased compared to DMSO control. These genes include, minichromosome maintenance proteins (MCM's), Cyclin E, CDK's, E2F's and Cdc2. Proteomic analysis also confirmed a decrease in proteins involved in cell cycle regulation. See FIG. 2(B). Thus, these transcriptomic and proteomic analyses coincide in that CB002-analog #4 reduces key regulators of the cell cycle. Taken with the fact that known xanthines such as caffeine deregulate the G2-checkpoint, the inventors examined the effects of the CB002-analogs on the cell cycle. These data show that the p53-restoring CB002-analog compounds, unlike known xanthines such as caffeine, pentoxifylline and theophylline, restore the p53 and do not deregulate the G2-checkpoint. Instead, treatment with these small molecule CB002-analogs results in activation of a S-phase DNA damage response pathway characterized by the increase in p-ATR^{(Thr1989)} and the inventors show this ultimately leads to a delay of cells in S-phase and this S-phase perturbation may contribute to cancer cell death. Importantly, the observed S-phase perturbation may lead to new therapeutic regimens such as synthetic lethality in BRCA-deficient cells and combination with PARP inhibitors.

ATF3/4 can regulate similar targets of that of p53, including p21. The inventors identified a small molecule PG3-Oc which involves the restoration of the p53 pathway independently of p53 through ATF4. Bracken et al., Trends Biochem. Sci., 29(8), 409-17 (2004). p53 was shown to indirectly repress many cell cycle genes through the induction of p21. P21 in turns binds to the DREAM repressor complex which represses genes controlled by E2Fs and CHR transcription factors. Fischer et al., Nucleic Acids Res, 44(13), 6070-86 (2016). Engeland, Cell Death Differ., 25(1), 114-132 (2018). The inventors observed many cell cycle genes downregulated at the transcriptional level relevant to the p53 signal pathway. This bioinformatic analysis predicted E2Fs as one of the transcription factors. The inventors have previously shown that CB002 induces p21 expression. Hernandez-Borrero et al., Cell Cycle, 17(5), 557-567 (2018). Combined with the analog #4 in this EXAMPLE, thus, the observed S-phase perturbation may be through p53-independent p21 stimulation that binds to DREAM complexes. Therefore, it will be interesting to see if ATF3/4 regulate p21 expression and the effect of p21 knockdown on cell cycle genes and effect on the S-phase perturbation observed by CB002 analogs.

The inventors show that CB002-analog #4 induces apoptosis in colorectal cancer patient-derived organoid cells and that it is safe both in vitro and in vivo as shown by lack of a statistically significant increase in the Sub-G1 population in normal human fibroblasts and also a healthy NSG mice body weight throughout treatment, respectively. The observed decrease in tumor volume was statistically significant at 5-weeks. This effect was suboptimal than desired. The decrease in tumor volume by CB002-analog #4 depends on Noxa. Because Noxa rarely is mutated in human cancer, its induction by the CB002-analogs offers a feasible therapeutic advantage leading to tumor cell death. Noxa expression can be a pharmacodynamic biomarker to predict therapeutic response.

Taken together, these data shows that CB002-analogs #4 and #10 represent a class of anti-tumor agents that provide a unique therapeutic strategy that can be clinically translated.

### Definitions

For convenience, the meaning of some terms and phrases used in the specification, examples, and appended claims, are listed below. Unless stated otherwise or implicit from context, these terms and phrases have the meanings below. These definitions are to aid in describing embodiments and are not intended to limit the claimed invention. Unless otherwise defined, all technical and scientific terms have the same meaning as commonly understood by a person having ordinary skill in the art to which this invention belongs. For any apparent discrepancy between the meaning of a term in the art and a definition provided in this specification, the meaning provided in this specification shall prevail.

A or An means at least one or one or more unless the context shows otherwise.

About means that the recited numerical value is approximate. Small variations would not significantly affect the practice of the disclosed embodiments. Where a numerical value is used unless shown otherwise by the context, about means the numerical value can vary by ±10% and remain within the scope of the disclosed embodiments.

Acylamino has the organic chemical art-recognized meaning of an amino group substituted by an acyl group.

Alkenyl has the organic chemical art-recognized meaning of a straight or branched alkyl group having 2 to 20 carbon atoms and having one or more double carbon-carbon bonds.

Alkoxy has the organic chemical art-recognized meaning of a straight or branched —O-alkyl group having 1 to 20 carbon atoms.

Alkyl has the organic chemical art-recognized meaning of a saturated hydrocarbon group which is straight-chained or branched.

Alkylamino has the organic chemical art-recognized meaning of an amino group substituted by an alkyl group. In some embodiments, the alkyl group is a lower alkyl group having from 1 to 6 carbon atoms.

Alkylene or Alkylenyl has the organic chemical art-recognized meaning of a divalent alkyl linking group.

Alkylthio has the organic chemical art-recognized meaning of an —S-alkyl group having from 1 to 6 carbon atoms.

Alkynyl has the organic chemical art-recognized meaning of a straight or branched alkyl group having 2 to 20 carbon atoms and one or more triple carbon-carbon bonds.

Amidino has the organic chemical art-recognized meaning of —C(═NH)NH₂.

Amino has the organic chemical art-recognized meaning of —NH₂.

Aminoalkoxy has the organic chemical art-recognized meaning of an alkoxy group substituted by an amino group.

Aminoalkyl has the organic chemical art-recognized meaning of an alkyl group substituted by an amino group.

Animal has the plain meaning and includes humans and non-human vertebrates, such as wild and domestic mammals.

Antagonize and Antagonizing have the organic chemical art-recognized meaning of reducing or eliminating one or more effects.

Aryl has the organic chemical art-recognized meaning of a monocyclic, bicyclic, or polycyclic aromatic hydrocarbon.

Arylalkyl has the organic chemical art-recognized meaning of an alkyl group substituted by an aryl. In some embodiments, an alkyl group is a C₁₋₆ alkyl group.

Arylamino has the organic chemical art-recognized meaning of an amino group substituted by an aryl group.

Arylene has the organic chemical art-recognized meaning of an aryl linking group, i.e., an aryl group that links one group to another in a molecule.

C₁₋₆-alkyl includes methyl, ethyl, propyl, C₄-alkyl, C₅-alkyl, and C₆-alkyl.

Cancer therapy has the medical art-recognized meaning of anticancer treatment to cure or prolong the life of a mammal with cancer, especially a human with cancer. Among the cancer therapies known in the medical art include: Some therapies treat tumors with mutated p53. Some chemotherapies involve administering 5-fluorouracil (5-FU), irinotecan, etoposide, gemcitabine, oxaliplatin, carboplatin, paclitaxel, or a combination thereof to the subject who has cancer. Some radiotherapies involve administering radiation to the subject who has cancer. Some chemotherapies involve administering PARP inhibitors. Some therapies target. DNA repair-deficient cancers that may have a defective repair of replicating DNA. Examples of DNA repair-deficient cancers include BRCA1-deficient cancers. Some chemotherapies involve administering immune checkpoint therapy, such as anti-PD-1, anti-PD-L1, or anti-CTLA4 antibodies. Some chemotherapies are targeted cancer therapies that involve administering anti-ATM, anti-ATR, anti-Chk1, anti-Chk2, anti-EGFR, anti-alk, anti-Her2, anti-NTRK, anti-BRAF, anti-KRAS antibodies to the subject who has cancer.

Carrier has the medical chemical art-recognized meaning of a diluent, adjuvant, or excipient with which a compound is administered in a composition.

CB002 has the organic chemical art-recognized meaning of a compound having the following structure:

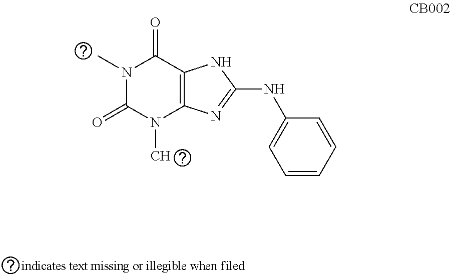 [SMILES: Cn1c(=O)c2[nH]c(Nc3ccccc3)nc2n(C)c1=O]

Compound has the medical chemical art-recognized meaning of all stereoisomers, tautomers, isotopes, and polymorphs of the compounds.

Comprising (and any form of comprising, such as Comprise, Comprises, and Comprised), Having (and any form of Having, such as Have and Has), Including (and any form of Including, such as Includes and Include), or Containing (and any form of Containing, such as Contains and Contain), are inclusive and open-ended and include the options following the terms, and do not exclude additional, unrecited elements or method steps.

Contacting has the medical chemical art-recognized meaning of bringing together two compounds, molecules, or entities in an in vitro system or an in vivo system.

Cyano has the organic chemical art-recognized meaning of —CN.

Cycloalkyl has the organic chemical art-recognized meaning of non-aromatic cyclic hydrocarbons, including cyclized alkyl, alkenyl, and alkynyl groups that have up to 20 ring-forming carbon atoms.

Darolutamide (DARO) is an androgen receptor (AR) antagonist approved to treat patients with non-metastatic CRPC. DARO has higher affinity to androgen receptor and preclinical activity against enzalutamide-resistant prostate cancer cell lines, including androgen receptor variants associated with enzalutamide agonism. Borgmann, Eur. Urol. (2018).

DR5 has the molecular biological art-recognized meaning of protein on the surface of some cells that binds another protein called TRAIL, which may kill some cancer cells. An increase in the amount or activity of DR5 on cancer cells may kill more cells. Also called death receptor 5, TRAIL receptor 2, TRAIL-R2, and tumor necrosis factor receptor superfamily member 10B. See National Cancer Institute (NCI) Dictionary of Cancer Terms.

ERK1/2 is a protein in the extracellular signal-regulated kinase 1/2 (ERK1/2) cascade, a central signaling pathway that regulates a wide variety of stimulated cellular processes, including proliferation, differentiation, and survival, but also apoptosis and stress response.

Halo has the organic chemical art-recognized meaning of halogen groups.

Haloalkoxy has the organic chemical art-recognized meaning of an —O-haloalkyl group.

Haloalkyl has the organic chemical art-recognized meaning of a C1-6alkyl group having one or more halogen substituents.

Heteroaryl has the organic chemical art-recognized meaning of an aromatic heterocycle having up to 20 ring-forming atoms (e.g., C) and having at least one heteroatom ring member (ring-forming atom) such as sulfur, oxygen, or nitrogen.

Heteroarylalkyl has the organic chemical art-recognized meaning of a C₁₋₆ alkyl group substituted by a heteroaryl group.

Heteroarylamino has the organic chemical art-recognized meaning of an amino group substituted by a heteroaryl group.

Heteroarylene has the organic chemical art-recognized meaning of a heteroaryl linking group, i.e., a heteroaryl group that links one group to another group in a molecule.

Heterocycle or heterocyclic ring has the organic chemical art-recognized meaning of a 5- to 7-membered monocyclic or 7- to 10-membered bicyclic ring system, any ring of which may be saturated or unsaturated, which ring consists of carbon atoms and from one to three heteroatoms chosen from N, O and S, wherein the nitrogen and sulfur heteroatoms may optionally be oxidized. The nitrogen heteroatom may optionally be quaternized, including by any bicyclic group in which any of the above-defined heterocyclic rings is fused to a benzene ring.

Heterocycloalkyl has the organic chemical art-recognized meaning of non-aromatic heterocycles having up to 20 ring-forming atoms, including cyclized alkyl, alkenyl, and alkynyl groups, where one or more of the ring-forming carbon atoms is replaced by a heteroatom, such as an O, N, or S atom. Hetercycloalkyl groups can be monocyclic or polycyclic (e.g., fused, bridged, or spiro systems).

Hydroxy or hydroxyl has the organic chemical art-recognized meaning of an —OH group.

Hydroxyalkyl or Hydroxylalkyl have the organic chemical art-recognized meaning of an alkyl group substituted by a hydroxyl group.

In need thereof has the medical chemical art-recognized meaning of that the individual, subject, or patient was identified as needing the method, prevention, or treatment. The identification can be by any diagnostic method. In any of the methods, preventions, and treatments described in this specification, the individual, subject, or patient can be in need. The individual, subject, or patient may be in an environment or will be traveling to an environment, or has traveled to an environment in which disease, disorder, or condition is prevalent.

Individual, Subject, and Patient used interchangeably means any animal. In some embodiments, the mammal is a human.

Integer means a numerical value that is a whole number. An integer from 1 to 5 means 1, 2, 3, 4, or 5.

Isolated has the medical chemical art-recognized meaning of that the compounds, or pharmaceutically acceptable salts thereof, are separated from other components of either (a) a natural source, such as a plant or cell, such as bacterial culture, or (b) a synthetic organic chemical reaction mixture, such as by conventional techniques.

Mammal has the medical chemical art-recognized meaning and includes rodents, monkeys, and humans. In some embodiments, the mammal is a human.

N-membered, where n is an integer, typically describes the number of ring-forming atoms in a moiety, where the number of ring-forming atoms is n. For example, pyridine is an example of a 6-membered heteroaryl ring, and thiophene is an example of a 5-membered heteroaryl ring.

Nitro has the organic chemical art-recognized meaning of —NO₂.

Optionally substituted has the organic chemical art-recognized meaning that a substitution is optional and, therefore, includes both unsubstituted and substituted atoms and moieties. A substituted atom or moiety shows that any hydrogen atom on the designated compound or moiety can be replaced with a selection from the mentioned substituent groups, provided that the normal valency of the designated compound or moiety is not exceeded and that the substitution results in a stable compound.

p53 pathway restoration has the cell-biological art-recognized meaning of a medical intervention effort to restore p53 activity as an anticancer therapeutic approach. See Martinez, Restoring p53 tumor suppressor activity as an anticancer therapeutic strategy. Future Oncol. 6(12), 1857-1862 (December 2010). The S-phase DNA damage response pathway is characterized by the increase in p-ATR^{(Thr1989)}. This increase ultimately leads to a delay in S-phase cells. This S-phase perturbation may contribute to cancer cell death.

p53, a tumor-suppressor, prevents cancer development via initiating cell-cycle arrest, cell death, repair, or anti-angiogenesis processes. Over 50% of human cancers harbor cancer-causing mutations in p53. p53 mutations abrogate its tumor-suppressor function and endow mutant p53 with a gain of function (GOF), creating a proto-oncogene that contributes to tumorigenesis, tumor progression, and chemotherapy or radiotherapy resistance. Targeting mutant p53 or restoring a wild-type p53 signaling pathway provides an attractive strategy for cancer therapy.

Pharmaceutically acceptable salts include but are not limited to salts of acidic or basic groups. Basic compounds can form a wide variety of salts with various inorganic and organic acids. Compounds that include an amino moiety may form pharmaceutically acceptable salts with various amino acids. Acidic compounds can form base salts with different pharmacologically acceptable cations. Salts include quaternary ammonium salts of the compounds described herein, where the compounds have one or more tertiary amine moiety.

Pharmaceutically acceptable has the medical chemical art-recognized meaning that the compounds, materials, compositions, or dosage forms are within the scope of sound medical judgment and are suitable for contact with tissues of humans and other animals. The pharmaceutically acceptable compounds, materials, compositions, or dosage forms result in no persistent detrimental effect on the subject or the general health of the treated subject. Still, transient effects, such as minor irritation or a stinging sensation, are common with the administration of medicament and are consistent with the composition, formulation, or ingredient, e.g., excipient, in question. Guidance as to what is pharmaceutically acceptable is provided by comparable compounds, materials, compositions, or dosage forms in the US Pharmacopeia or another generally recognized pharmacopeia for use in animals, and more particularly in humans.

Phenyl has the organic chemical art-recognized meaning of —C₆H₅. A phenyl group can be unsubstituted or substituted with one, two, or three suitable substituents.

Prevention or preventing has the medical chemical art-recognized meaning of reducing the risk of acquiring a disease, condition, or disorder.

Prodrug has the medical chemical art-recognized meaning of a derivative of a known direct-acting drug, which may have enhanced delivery characteristics and therapeutic value compared to the active drug. It is transformed into an active drug by an enzymatic or chemical process. Prodrugs can be prepared by modifying functional groups present in the compounds so the modifications are cleaved, either in routine manipulation or in vivo, to the parent compounds.

Purified has the medical chemical art-recognized meaning that when isolated, the isolate contains at least 90%, at least 95%, at least 98%, at least 99%, or 100% of a compound by weight of the isolate.

Quaternary ammonium salts have the medical chemical art-recognized meaning of derivatives of the disclosed compounds with one or more tertiary amine moieties wherein at least one of the tertiary amine moieties in the parent compound is modified by converting the tertiary amine moiety to a quaternary ammonium cation via alkylation.

S-phase perturbation means disruption or delay of the typical passage of cells through the S-phase of the cell cycle during which DNA replication occurs (where the DNA content of the cell doubles from 1N content (haploid) to 2N content (diploid). This can occur through activation of an S-phase checkpoint, representing a response to cellular sensing of disrupted DNA replication or disrupted cell signaling of processes important to pass cells through the S-phase of the cell cycle (during which DNA replication occurs.

Solubilizing agent has the medical chemical art-recognized meaning of agents that result in the formation of a micellar solution or a true solution.

Solution/suspension has the medical chemical art-recognized meaning of a liquid composition wherein a first portion of the active agent is present in the solution. A second portion of the active agent is present in particulate form, in suspension in a liquid matrix.

Substantially isolated has the medical chemical art-recognized meaning of a compound that is at least partially or substantially separated from the environment in which it is formed or detected.

Suitable substituent or substituent has the medical chemical art-recognized meaning of a group that does not nullify the synthetic or pharmaceutical utility of the compounds or the intermediates useful for preparing them. A person having ordinary skill in medical chemical art can readily choose a suitable substituent based on the stability and pharmacological and synthetic activity of the compounds described herein.

Therapeutically Effective amount has the medical chemical art-recognized meaning of the amount of active compound or pharmaceutical agent that elicits the biological or medicinal response sought in a tissue, system, animal, individual, or human by a researcher, veterinarian, medical doctor, or another clinician. The therapeutic effect depends upon the disorder being treated or the biological effect desired. The therapeutic effect can be a decrease in the severity of symptoms associated with the disorder or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the subjects response to treatment.

Treat, Treated, or Treating has the medical chemical art-recognized meaning of both treatment and prophylactic or preventive measures wherein the object is to prevent or slow down (lessen) an undesired physiological condition, disorder or disease, or obtain beneficial or desired clinical results. Beneficial or desired clinical results include, but are not limited to, alleviation of symptoms; diminishment of the extent of the condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of the condition, disorder or disease. Treatment includes eliciting a clinically significant response, optionally without excessive levels of side effects. Treatment also includes prolonging survival as compared to expected survival if not receiving treatment.

The terms Comprise and Comprising should be interpreted as referring to elements, components, or steps in a non-exclusive manner, indicating that the referenced elements, components, or steps may be present, used, or combined with other elements, components, or steps. The singular terms a, an, and the include plural referents unless context shows otherwise. Similarly, the word or should cover and unless the context shows otherwise. The abbreviation e.g. is used to show a non-limiting example and is synonymous with the term for example.

When a range of values is provided, each intervening value, to the tenth of the unit of the lower limit, unless the context dictates otherwise, between the upper and lower limit of that range and any other stated or intervening value in that range of values.

### Materials, Methods, and Assays

A person of ordinary skill in the medical chemical art can use these assays as guidance to predictable results when making and using the invention.

Cell-based drug screening for p53 pathway-restoring small molecules materials and methods. The high-throughput screening was performed using a noninvasive bioluminescence imaging in human colorectal cancer cell lines that stably express a p53-regulated luciferase reporter. Wang, Kim, & El-Deiry, Proc. Natl. Acad. Sci. USA, 103(29), 11003-8 (2006). Cells were seeded in 96-well plates (Greiner Bio-One) at a density of 1×10⁴ cells per well. p53 transcriptional activity was imaged using an IVIS imaging system for 3-4 hours. Alternatively, cells were treated with the compound from 0 to 600 μM. p53 transcriptional activity was imaged using an IVIS imaging system at six hours.

CB002 analog screening (additional method). CB002 structural analogs were obtained from Chembridge Library. The screening was performed in the human SW480 colorectal cancer cell line that stably expresses a p53-regulated luciferase reporter. Cells were seeded at a density of 1×10⁴ cells per well in 96-well plates, commercially available from Greiner Bio-One North America, Monroe, N.C., USA. p53 transcriptional activity was imaged using an IVIS imaging system at six hours.

Cell lines and culture conditions. DLD-1 (p53^{S241F}), SW480 (p53^{R273H,P309S}), and HCT116 (p53^{WT}) p53^{-/-} colorectal cancer cell lines that stably express a p53-regulated luciferase reporter were generated, as shown in U.S. Pat. Publ. 2019/0225613 A1 (El-Diery et al.). See also Hernandez-Borrero et al., Cell Cycle, 17(5), 557-567 (2018). DLD-1, SW480, and HCT116 colorectal cancer cell lines and WI38 normal lung fibroblast cells can be purchased from the American Type Culture Collection (ATCC), Manassas, Va., USA. HCT116 p53^{-/-} was obtained from the Vogelstein Laboratory, Johns Hopkins University. RXF393 renal cancer cell lines, WI38 normal lung cell fibroblasts, and MRCS normal lung cell fibroblasts can be purchased from American Type Culture Collection. Cell lines were maintained in HyClone™ Dulbecco's High Glucose Modified Eagles Medium (DMEM, GE Healthcare, La Palma, Calif., USA), HyClone™ McCoy's 5A (GE Healthcare, La Palma, Calif., USA), HyClone™ RPMI 1640 (GE Healthcare, La Palma, Calif., USA), or Eagle's Minimum Essential Medium (EMEM, ATCC) containing 10% fetal bovine serum and 1% penicillin/streptomycin (complete media) at 37° C. in 5% CO₂, as recommended by ATCC.

Cell lines and culture conditions (additional method)., HCT116 R175H p53, and HCT116 R273H p53. The SW480 cancer cell line stably expresses a p53-regulated luciferase reporter. Cell lines were authenticated and tested for mycoplasma. Cell lines were maintained in HyClone™ Dulbecco's High Glucose Modified Eagles Medium (DMEM, GE Healthcare, La Palma, Calif., USA), HyClone™ McCoy's 5A (GE Healthcare, La Palma, Calif., USA) or Eagle's Minimum Essential Medium (EMEM, ATCC, Manassas, Va., USA) containing 10% fetal bovine serum and 1% penicillin/streptomycin (complete media) at 37° C. in 5% CO₂, as recommended by ATCC, Manassas, Va., USA.

CellTiter-Glo® luminescent cell viability assay. Cells were seeded in 96-well plates at a density of 5×10³ cells per well. 20 μL of CellTiter-Glo® Reagent was added directly to the wells, following the manufacturers protocol. The bioluminescence signal was determined using an IVIS imaging system, optionally at a period of 48-72 hours after treatment.

CellTiter-Glo® luminescent cell viability assay (additional method). Cell lines at a concentration of 4×10³ cells/well were seeded out on an opaque 96-well plate and treated with CB002 and CB002-analog #11 in ranging doses starting from 200 μmol/L with dimethyl sulfoxide controls. At seventy-two hours after treatment, cells were mixed with 30 μL Cell Titer-Glo reagent. After ten minutes of room temperature incubation, the cells were imaged using an IVIS imaging system commercially available from Xenogen Corporation, Alameda, Calif., USA.

Knockdown expression of p73, DR5, ATG5, or FADD using siRNA. A total of 1×10⁵ cells/well were plated per well in a 12-well plate in medium with 10% FBS without antibiotic. Forward transfection of p73 siRNA (s14319, Ambion®, Ambion Inc., Austin, Tex., USA), DR5 (sc-40237, Santa Cruz Biotechnology), atg5 (137766, Ambion®, Ambion Inc., Austin, Tex., USA), or FADD (S16706, Ambion®, Ambion Inc., Austin, Tex., USA) was performed using the Lipofectamine® RNAiMAX Transfection Reagent (Life Technologies) and incubated for forty-eight hours before treatment, e.g., before drug treatments.

Overexpression of p53 R175H mutant by lentivirus infection. HCT116 p53^{-/-} cells, obtained from the Vogelstein Laboratory, Johns Hopkins University, were infected with a lentivirus vector containing the p53 R175H mutant (pLenti6/V5-p53_R175H, Addgene). Cells were selected with blasticidin (8 μg/mL)-containing media cultured for ten days. Blasticidin-resistant clones (pooled clones) were screened for expression of the p53 R175H mutation by Western blot analysis with p53 DO-1 antibody.

Knockdown expression of NOXA by lentivirus infection. A NOXA shRNA plasmid construct was amplified according to the manufacturers recommendation (TRC Lentiviral Human PMAIP1 shRNA, Dharmacon). Plasmid DNA was isolated using the PureLink® HiPure Plasmid Filter Maxiprep Kit (Invitrogen) according to the manufacturers instructions. Lentivirus production was performed by transfecting HEK293T cells at a density of 8×10⁶ cells per 10-cm dish with 1.6 μg pMD2.G envelope plasmid, 3.2 μg psPAX2 packaging vector, 3.2 μg plasmid DNA, and 24 μL of Lipofectamine® Transfection reagent 2000 (Life Technologies) in a total volume of the reaction of 1 mL of antibiotic-free DMEM media for 6-10 hours. The media was then replaced with antibiotic-free DMEM. Lentiviral particles were collected between forty-eight to seventy-two hours. SW480 cells (2.3×10⁶ cells per well in a 12-well plate) were infected 1:1 (virus-containing media: antibiotic-free DMEM media, total volume 1 mL) for twenty-four hours. Then, the media was replaced with DMEM complete media for an additional twenty-four hours. Cells were split and seeded (20% confluent) for selection in a 10 cm dish with puromycin (2.5 μg/mL)-containing complete DMEM media and cultured for ten days. Puromycin containing complete media was replaced every 2-3 days. Puromycin-resistant clones were screened for knockdown of NOXA by Western blot analysis with NOXA antibody.

Colony formation assay. Cells were seeded in 6-well plates at a density of 500 cells per well. Cells were treated with CB002 small molecules for twenty-four hours. Then, cells were cultured in complete drug-free media for fifteen days. During the fifteen days, the media was changed every two-three days. At the end of the two weeks, the media was removed. Wells were washed twice with Dulbecco's phosphate-buffered saline (PBS). The colonies were fixed and stained with 10% methanol and 0.25% crystal violet (Sigma-Aldrich, St. Louis, Mo., USA) for thirty minutes. Wells were then carefully washed with distilled, deionized water and allowed to dry.

Apoptosis assay. Apoptotic cells were quantified by sub-G1 analysis. Cells were seeded at a density of 2.5×10⁵to 5×10⁵in a 6-well plate and treated forty-eight to seventy-two hours. After treatment, adherent cells were trypsinized and collected along with floating cells, washed with phosphate-buffered saline, and fixed in 70% ethanol. Cells were then incubated in a phosphate-citric acid buffer (0.2M Na₂HPO₄+0.1M citric acid, pH 7.8) at room temperature for five minutes, spun down, and resuspended for staining with 50 μg/mL propidium iodide (PI) in the presence of 250 μg/mL pancreatic ribonuclease (RNase A). Sub-G1 analyses were performed using an Epics Elite Epics flow cytometer (Coulter-Beckman).

Immunoblotting. After treatment, cells were harvested by trypsinization, washed with phosphate-buffered saline, and lysed with RIPA buffer (Sigma-Aldrich) for thirty minutes to one hour at 4° C. Protein lysates were spun down. The supernatant was collected. Protein quantification was performed using a Pierce™ BCA Protein Assay Kit (Thermo Fisher Scientific, Waltham, Mass., USA). 1× NuPAGE® LDS sample buffer (Thermo Fisher Scientific, Waltham, Mass., USA) and 2-Mercaptoethanol as the reducing agent (Sigma-Aldrich) were added to protein lysates followed by boiling for fifteen minutes at 95° C. Equal total protein amounts samples were loaded into NuPAGE™ Novex™ 4-12% Bis-Tris Protein Gels (1.5 mm, Thermo Fisher Scientific, Waltham, Mass., USA). Gel electrophoresis was performed with NuPAGE™ MES SDS Running Buffer. Proteins were transferred onto an Immobilon-P membrane (PVDF, EMD Millipore, Taunton, Mass., USA) using a Bio-Rad system with a 10% Tris-Glycine and 10% methanol transfer buffer diluted in distilled and deionized water. After transfer, membranes were blocked with 10% milk in Tris-buffered saline with Tween 20 (TBST) solution. The membranes were then incubated overnight with the primary antibody, washed with TBST, and incubated with secondary antibody for one hour. Incubations were performed in 5% milk in TBST solution. Signal was detected by using a chemiluminescent detection kit, followed by autoradiography. The following antibodies were used: p53 (DO-1, 1:1000, Santa Cruz, Calif., USA), p73 (1:1000, Bethyl Laboratories), p21, and NOXA (1:250, EMD Millipore, Taunton, Mass., USA), DR5, FADD, cleaved caspase 3, cleaved caspase 8, cleaved PARP, and LC3B (1:1000, Cell Signaling Technology, Inc., Danvers, Mass. USA), and β-actin (1:10000, Sigma-Aldrich, St. Louis, Mo., USA).

Immunoblotting (additional method). After treatment, floating cells were collected, and adherent cells were trypsinized, washed with phosphate-buffered saline, and lysed with RIPA buffer (Sigma-Aldrich, St. Louis, Mo., USA) for thirty minutes to one hour at 4° C. Protein lysates were pelleted. The supernatant was collected. Total protein per sample was determined using a Pierce™ BCA Protein Assay Kit (Thermo Fisher Scientific, Waltham, Mass., USA). Proteins were denatured using 1× NuPAGE® LDS sample buffer (Thermo Fisher Scientific, Waltham, Mass., USA) and reduced with 2-mercaptoethanol (Sigma-Aldrich, St. Louis, Mo., USA). Protein lysates were boiled for fifteen min at 95° C. After protein normalization, samples were loaded into NuPAGE™ Novex™ 4-12% Bis-Tris Protein Gels (Thermo Fisher Scientific, Waltham, Mass., USA). Gel electrophoresis was performed with NuPAGE™ MES SDS Running Buffer. Proteins were transferred onto an Immobilon-P membrane (PVDF, EMD Millipore, Taunton, Mass., USA) using a Bio-Rad system with a 10% Tris-Glycine and 10% methanol transfer buffer diluted in distilled and deionized water. Membranes were blocked with 10% milk in Tris-buffered saline with Tween 20 (TBST) solution. The membranes were incubated overnight with the primary antibody, washed with TBST, and incubated with secondary antibody for one hour. Primary antibody incubations were performed in 5% milk or 5% bovine serum albumin in TBST solution as per manufacturer instructions. Signal was detected using a chemiluminescent detection kit, followed by autoradiography. The following antibodies were used: p53 (1:1000, #sc-126, Santa Cruz Biotechnology, Santa Cruz, Calif., USA), p73 (1:1000, #A300-126A, Bethyl Laboratories), Noxa (1:250, #OP180, EMD Millipore, Taunton, Mass., USA), DR5 (#3696), cleaved PARP (#9546), phospho-RPA32/RPA2 (#54762), p-cdc2 (#9111), p-cdc25c, pH3 (#3377) (1:1,000; Cell Signaling Technology, Inc., Danvers, Mass. USA) and β-actin (1:10000, A5441, Sigma).

Western immunoblot analysis (additional method). Proteins were isolated using NP40 Lysis Buffer (20 mmol/L Tris-HCl (pH 7.4), 150 mmol/L NaCl, 5 mmol/L EDTA, 50 mmol/L NaF, 1 mmol/L glycerophosphate, 5 mmol/L Na₄P₂O₇, 0.5% NP40, and complete protease inhibitor cocktail (Roche)) and electrophoresed through 4-12% SDS-PAGE followed by semi-dry transfer to PVDF membranes. The PVDF membranes were incubated with different antibodies, including p21 (OP64-100UG, EMD Millipore, Taunton, Mass., USA), DR5 (3696S, Cell Signaling Technology, Inc., Danvers, Mass. USA), p53(sc-126, Santa Cruz Biotechnology, Santa Cruz, Calif., USA), and RAN (610341, BD Transduction Laboratories) in blocking buffer at 4° C. overnight. The bound antibody is detected using IRDye secondary antibodies (LI-COR Biosciences) in Odyssey blocking buffer for one hour, then imaged using the ODYSSEY infrared imaging system.

Bioluminescence assay. Cell-based screening of p53 transcriptional activity for small molecule CB002 was accomplished using noninvasive bioluminescence imaging in human colorectal cancer cell lines SW480, DLD-1, DLD-1 p73^{-/-}, HCT116, and HCT116 p53^{-/-}. These cell lines stably express a p53 reporter, PG13-luc. Cells were seeded in the opaque 96-well culture at a density of 5×104 cells/well. The cells were treated with CB002 at ranging doses with dimethyl sulfoxide controls. Bioluminescence in cells was imaged for p53 transcriptional activity at two hours and twenty-four hours using an IVIS imaging system commercially available from Xenogen Corporation, Alameda, Calif., USA.

FACS assay. Cells were seeded out at 1×10⁶ cells/well on six-well plates and treated with CB002 and CB002-analog #11 at ranging doses with dimethyl sulfoxide controls. Cells were harvested after seventy-two hours of treatment. All cells, including floating cells, were fixed with ethanol and stained with propidium iodide. The cells were analyzed using the Epics Elite flow cytometer to measure the DNA content of the stained cells.

Cell synchronization. Synchronized cells were synchronized by double thymidine block. Cells were treated with 2 μM thymidine for sixteen hours. The drug was removed and replaced by complete growth media for eight hours. Cells were treated for the second time with 2 μM thymidine for sixteen hours. At this point, cells were treated and harvested.

Propidium iodide and BrdU flow cytometry assay. Cells were seeded at a density of 5×10⁵ in a 6-well plate and treated for forty-eight to seventy-two hours. After treatment, floating cells were collected, and adherent cells were trypsinized, pelleted, washed with phosphate-buffered saline, and fixed in 70% ethanol overnight. For propidium iodide (PI) based Sub-G1 apoptosis analysis, cells were spun down after fixation and resuspended in phosphate-citric acid buffer (0.2M Na2HPO4+0.1M Citric Acid, pH 7.8) at room temperature for five minutes. The cell pellet was resuspended for staining with 50 μg/mL PI and 250 μg/mL ribonuclease (RNase A). For BrdU analysis, after cell fixation, cells were spun down and resuspended in one mL of 2N HCL with 0.5% Triton X-100 for thirty minutes at room temperature. Cells were pelleted, washed with phosphate-buffered saline, and resuspended in 20 μL BrdU antibody (BD Biosciences, cat no. 347580) diluted in 0.5% Tween 20/PBS/5% bovine serum albumin for thirty minutes at room temperature. Cells were then spun down and resuspended in 140 μg/mL goat-anti mouse Alexa Fluor 488 (#A-11008, Thermo Fisher Scientific, Waltham, Mass., USA) in 0.5% Tween 20 in phosphate-buffered saline/5% bovine serum albumin for thirty minutes at room temperature. Cells were then spun down and resuspended in 5 μg/mL PI: 250 μg/mL RNase A solution. Samples were analyzed on an Epics Elite Epics flow cytometer (Coulter-Beckman).

BrdU analysis gating. Cell aggregates were gated out in the PI Peak vs DNA PI histogram. BrdU lower limit intensity was set on the upper limit of the negative control. The no BrdU antibody assays and no goat-anti mouse Alexa Fluor 488 antibody assays were the negative controls. Haploid cell gating shows the haploid BrdU-positive cells. S-phase and G2-phase boundaries were determined by propidium iodide staining that showed G1 and G2 as per DNA content. Gating was held constant throughout the samples within a given assay.

Microarray analysis. SW480 cells were seeded at a density of 1×10⁶ in 10 cm dishes. After being adhered, the cells were treated with dimethyl sulfoxide vehicle control or CB002-analog #4. Floating cells were collected, and adherent cells were trypsinized at twelve hours of treatment. The cells were pelleted. According to manufacturer instructions, RNA was isolated from the cells using a Quick-RNA™ MiniPrep (#R1055, Zymo Research Irvine, Calif., USA). RNA quality was tested using an Agilent Bioanalyzer RNA kit. When the RNA quality was enough, RNA was amplified and labeled using the low RNA input linear amplification kit commercially available from Agilent Technologies, Santa Clara, Calif., USA. Labeled cDNA was hybridized onto Affymetrix Human Gene 2.0-ST array. Significant changes in gene expression were determined at 1.5-fold either up or down in dimethyl sulfoxide vehicle control versus CB002-analog #4.

Proteomic analysis. SW480 cells were seeded at a density of 1×10⁶ in 10 cm dishes and treated with dimethyl sulfoxide vehicle control or CB002-analog #4 for twenty-four hours. Floating cells were collected. Adherent cells were trypsinized. Cells were spun down and washed with phosphate-buffered saline. The pelleted cells were frozen and submitted for LC-MS/MS identification and comparative analysis at the Center for Cancer Research Development, Proteomics Core Facility at the Rhode Island Hospital. Samples (cell pellets) were lysed with a lysis buffer (8M urea, 1 mM sodium orthovanadate, 20 mM HEPES, 2.5 mM sodium pyrophosphate, 1 mM β-glycerophosphate, pH 8.0, twenty minutes, 4° C.) followed by sonication at 40% amplification by using a microtip sonicator (QSonica, LLC, Model no. Q55) and cleared by centrifugation (14,000×g, fifteen minutes, 15° C.). Protein concentration was measured (Pierce BCA Protein Assay, Thermo Fisher Scientific, Waltham, Mass., USA). A total of 100 μg of protein per sample was subjected to trypsin digestion. Tryptic peptides were desalted using C18 Sep-Pak plus cartridges (Waters, Milford, Mass., USA) and were lyophilized for forty-eight hours to dryness. The dried peptides were reconstituted in buffer A (0.1M acetic acid) at a concentration of 1 μg/μl, and 5 μl was injected for each analysis.

Sample preparation for LC-MS/MS analysis. SW480 cells were seeded at a density of 1×10⁶ in 10 cm dishes and treated with DMSO vehicle control or CB002-analog #4 for twenty-four hours. Floating cells were collected and adherent cells were trypsinized. Cells were spun down, wash with PBS and pelleted cells were flash frozen with liquid N2 and subjected for LC-MS/MS analysis. Briefly, cell pellets were lysed with a lysis buffer (8M urea, 1 mM sodium orthovanadate, 20 mM HEPES, 2.5 mM sodium pyrophosphate, 1 mM β-glycerophosphate, pH 8.0, 20 min, 4° C.) followed by sonication at 40% amplification by using a microtip sonicator (QSonica, LLC, Model no. Q55) and cleared by centrifugation (14,000×g, 15 minutes, 15° C.). Protein concentration was measured (Pierce BCA Protein Assay, Thermo Fisher Scientific) and 100 μg of protein per sample was subjected for trypsin digestion. Tryptic peptides were desalted using C18 Sep-Pak plus cartridges (Waters, Milford, Mass., USA) and were lyophilized for 48 hours to dryness. The dried peptides were reconstituted in buffer A (0.1M acetic acid) at a concentration of 1 μg/μL and 5 μL was injected for each analysis.

LC-MS/MS analysis. LC-MS/MS was performed on a fully automated proteomic technology platform that includes an Agilent 1200 Series Quaternary HPLC system, commercially available from Agilent Technologies, Santa Clara, Calif., USA, connected to a Q Exactive Plus mass spectrometer commercially available from Thermo Fisher Scientific, Waltham, Mass., USA. The LC-MS/MS setup was used as described by Ahsan et al., J. Proteomics, 165, 69-74 (2017). The peptides were separated through a linear reversed-phase 90 min gradient from 0% to 40% buffer B (0.1M acetic acid in acetonitrile) at a flow rate of 3 μl/min through a 3 μm 20 cm C18 column (OD/ID 360/75, Tip 8 μm, New Objectives, Woburn, Mass., USA) for 90-minute runtime. The electrospray voltage of 2.0 kV was applied in a split-flow configuration. Spectra were collected using a top-9 data-dependent method. Survey full-scan MS spectra (m/z 400-1800) were acquired at a resolution of 70,000 with an AGC target value of 3×106 ions or a maximum ion injection time of 200 milliseconds. The peptide fragmentation was performed via higher-energy collision dissociation with the energy set at 28 normalized collision energy (NCE). The MS/MS spectra were acquired at a resolution of 17,500, with a targeted value of 2×104 ions or a maximum integration time of 200 milliseconds. The ion selection abundance threshold was set at 8.0×10² with charge state exclusion of unassigned and z=1, or 6-8 ions and dynamic exclusion time of thirty seconds.

Immunohistochemistry. 30,000 cells/well were seeded in 8-chamber slides. Cells were washed with phosphate-buffered saline at the harvesting time point and fixed with 4% parafornaldehyde for 25 minutes. Cells were then washed with phosphate-buffered saline and permeabilized with 0.2% Triton X-100 for 5-10 minutes. Cells were then washed with phosphate-buffered saline and incubated overnight 1:100 with the primary antibody Cyt-C (#sc-13560, Santa Cruz Biotechnology, Santa Cruz, Calif., USA), Tom-20 (#42406), commercially available from Cell Signaling Technology, Inc., Danvers, Mass. USA), cells were washed with phosphate-buffered saline and incubated with secondary antibody 1:200 goat-anti mouse Alexa Fluor 488 (#A-11008, Thermo Fisher Scientific, Waltham, Mass., USA) and Cy3 AffiniPure Donkey anti-rabbit (#711-165-152, Jackson ImmunoResearch, West Grove, Pa., USA) for one hour followed by phosphate-buffered saline washing, 1:400 Dapi staining, washed with phosphate-buffered saline, and imaged. Organoid viability imaging was determined by CellTrace™ Calcein Green (#C34852, Thermo Fisher Scientific, Waltham, Mass., USA), Ethidium Homodimer-1 (#E1169, Thermo Fisher Scientific, Waltham, Mass., USA), Ki67 incubated at 37° C. for one hour then washed with phosphate-buffered saline and imaged. Imaging was done using a Leica Confocal Microscope.

Drug efficacy using in vivo tumor xenografts. In vivo drug efficacy studies were performed on ten NSG female randomized mice per cohort. The mice had tested negative for any pathogen. Tumor inoculation was induced by subcutaneous injection in the left and right dorsal flank, each with a 150 μL suspension of 1-5×10⁶ human colon cancer cells in phosphate-buffered saline with Matrigel (1:1). After tumor size reached 100 mm³, mice were treated 3×/week with dimethyl sulfoxide vehicle or CB002-analog #4 via oral gavage (22-gauge needle) in a solution of 10% dimethyl sulfoxide, 20% Kolliphor® EL (Sigma cat. no. C5135), and 70% phosphate-buffered saline. Mouse weight and tumor measurements were recorded one-two times per week. Tumor volume was calculated as V=0.5*L*W\hat{ }2, where L is length and W is the width of the tumor. At the end of the assay, mice were euthanized by CO₂. All in vivo procedures were performed according to an approved Institutional Animal Care and Use Committee (IACUC) protocol.

Database search and label-free quantitative analysis. Peptide spectrum matching of MS/MS spectra of each file was searched against the NCBI Human database (TaxonID: 9606) using the Sequest algorithm within Proteome Discoverer v 2.4 software (Thermo Fisher Scientific, Waltham, Mass., USA). The Sequest database search was performed with these parameters: trypsin enzyme cleavage specificity, two possible missed cleavages, 10 ppm mass tolerance for precursor ions, 0.02 Da mass tolerance for fragment ions. Search parameters permitted dynamic modification of methionine oxidation (+15.9949 Da) and static modification of carbamidomethylation (+57.0215 Da) on cysteine. Peptide assignments from the database search were filtered down to a 1% FDR. The relative label-free quantitative and comparative among the samples were performed using the Minora algorithm and the adjoining bioinformatics tools of the Proteome Discoverer 2.4 software. To select proteins that show a statistically significant change in abundance between two groups, a threshold of 1.5-fold change with a p-value (0.05) was selected.

Statistical analysis. Data are presented as means±SEM (three biological replicates). Two-way ANOVA for two comparisons was performed, with p<0.05 defined as statistically significant. Comparisons were made against dimethyl sulfoxide vehicle control. Two-way ANOVA or unpaired t-test for two comparisons was also performed to assess the statistical significance, with p<0.05 defined as statistically significant. Data are presented as means±SEM (three biological replicates). Comparisons were made against the dimethyl sulfoxide vehicle control.

The following EXAMPLE is provided to illustrate the invention and should not be considered to limit its scope.

### EXAMPLE 1

A Subgroup of Potent CB002 xanthine Derivatives Define a Class of Anticancer Drugs that Restore the p53 Pathway in Tumors with Mutated p53 and Target an S-Phase Checkpoint.

The inventors previously discovered the parental compound CB002 and its analogs capable of restoring the p53-pathway in tumors with mutated p53. U.S. Pat. Publ. 2019/0225613 A1. These compounds are xanthine analogs, but unlike known xanthines such as caffeine, pentoxifylline, and theophylline, they restore the p53 pathway, including upregulation of Noxa, but do not deregulate the G2 checkpoint. A 24-hour treatment in DLD-1 and SW480 cells shows that the analogs induce p53 transcriptional targets, whereas caffeine, pentoxifylline, and theophylline do not.

The cell cycle analysis data in synchronized SW480 cells treated with etoposide shows that CB002 and its analogs perturb the S-phase of the cell cycle and do not deregulate the G2 checkpoint. This perturbation is also shown by increased p-RPA/RPA2 and cyclin E and decreased p-histone 3 protein expression. The microarray data show that the p53-pathway restoring xanthine analogs downregulate essential proteins of the DNA synthesis and repair machinery. The inventors are investigating the involvement of critical kinases and transcriptional factors regulated in the S-phase following treatment with CB002 and its more potent analogs. These data show the activation of ATR in response to CB002 analog treatment in SW480 cells at twenty-four hours. The inventors are evaluating the benefit of synthetic lethality of these analogs in BRCA-deficient cell lines, focusing on target identification, determining the proteomic changes, and in vivo efficacy of the p53-pathway restoring CB002 xanthine analogs.

CB002 analog xanthine derivatives provide a unique therapeutic strategy that can be clinically translated.

### EXAMPLE 2

CB002-Analog xanthine Derivatives Restore p53-Pathway and Target S-Phase Checkpoint.

Results. The inventors evaluated potent CB002-analog compounds. The inventors identified a mechanism of action of a class of anticancer drugs based upon the results. Based on their molecular structure as xanthine derivatives, the identified class of CB002-analog xanthine derivatives do not deregulate the G2-checkpoint.

The CB002-analog xanthine derivatives described in this specification perturb S-phase and restore the p53-pathway, a property not found with caffeine, pentoxifylline, and theophylline. The inventors further characterized and defined the new class of small molecules with anti-tumor properties by transcriptomic and proteomic analysis. CB002 and structural analogs restore the p53 pathway while xanthines such as caffeine, pentoxifylline, and theophylline do not.

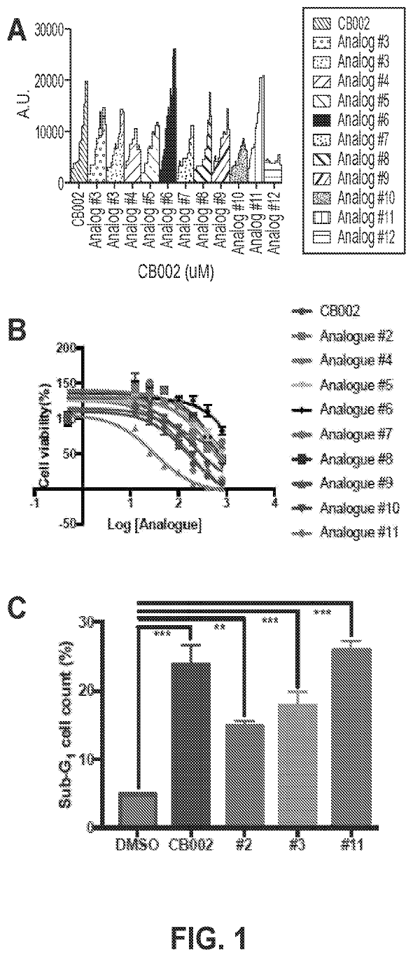

The inventors next identified more potent analogs of parental compound CB002. The inventors tested CB002 analogs in the Chembridge library for the capability to induce the luciferase activity using a p53-reporter assay and also determined the IC₅₀ values for the compounds by a Cell-Titer glow cytotoxicity assay. See FIG. 1. Most of the CB002-analogs tested, except for analog #12, enhanced p53-reporter activity. The inventors investigated the capability of a set of the CB002-analogs to induce apoptosis, as shown by propidium iodide (PI) staining Sub-G1 population. The treatment of tumor cells with CB002-analogs resulted in a significant increase in Sub-G1 content.

The inventors investigated the capability of a set of the CB002-analogs to induce apoptosis as shown by propidium iodide (PI) staining Sub-G1 population. As shown in FIG. 1(C), treatment of tumor cells with CB002-analogs at an IC₅₀ concentration (100 μM) resulted in a significant increase in Sub-G1 content in SW480 cells. The most potent CB002-analog #4, increases cleaved-PARP and cytochrome C release from the mitochondria to the cytosol providing further evidence for apoptosis induction in SW480 tumor cells. The inventors investigated whether the p53-family member p73 may be a mediator of apoptosis and responsible for inducing p53 transcriptional targets by CB002-analogs.

CB002 and structural analogs induce Noxa in an ATF3/4-dependent manner, independent of p73. The most potent CB002-analog #4 increases cleaved-PARP and cytochrome C release from the mitochondria to the cytosol providing further evidence for apoptosis induction in tumor cells. The inventors investigated whether the p53-family member p73 may be a mediator of apoptosis and responsible for inducing p53 transcriptional targets by CB002-analogs. p53-targets Noxa and DR5 were induced independently of p73. PARP cleavage occurred despite effective p73 knockdown in CB002-analog #4 treated tumor cells. The inventor's previously published CB002 data showed that Noxa plays a crucial role in mediating CB002-induced apoptosis.

The inventors assayed whether CB002 analogs induce Noxa expression in four human colorectal cancer cell lines. In DLD-1, SW480, HCT116, and HCT116 p53^{-/-} tumor cells expressing the exogenous R175H p53 mutant, Noxa protein expression was induced inventors observed some variation across cell lines.

The inventors investigated whether other known xanthine derivatives, i.e., caffeine, pentoxifylline, and theophylline, can induce Noxa expression. The inventors found that only the p53-pathway restoring CB002-analog xanthine compounds and not caffeine, pentoxifylline, and theophylline induce Noxa protein expression.

The inventors previously showed for CB002, p53-targets Noxa and DR5 were induced independently of p73 and PARP cleavage occurred despite effective p73 knockdown in CB002-analog #4 treated SW480 tumor cells. Hernandez-Borrero et al., (2018). The inventors' previously published CB002 data showed that Noxa plays a key role in mediating CB002-induced apoptosis. Hernandez-Borrero et al., (2018). Therefore, the inventors next determined if CB002 analogs induce Noxa expression in 4 human colorectal cancer cell lines. In DLD-1 (p53^{S241F}), SW480(p53^{R273H,P309S}), HCT116(p53^{WT}), and HCT116 p53^{-/-} tumor cells expressing the exogenous R175H p53 mutant, Noxa protein expression was induced, though some variation across cell lines was observed. The inventors nest investigated whether other known xanthine derivatives, i.e., caffeine, pentoxifylline and theophylline can induce Noxa expression. The inventors found that only the p53-pathway restoring CB002-analog xanthine compounds and not caffeine, pentoxifylline and theophylline, induce Noxa protein expression. Because Noxa can be transcriptionally activated independently from p53, the inventors investigated other transcription factors involved in Noxa induction. The inventors performed a knockdown of integrated stress response transcription factors ATF3/4 on SW480 cells. Knockdown of ATF3/4 upon treatment with 100 μM CB002 or 25 μM CB002-analog #4 abrogated Noxa protein induction. Thus, the data shows that ATF3/4 μlays a role in regulating Noxa expression.

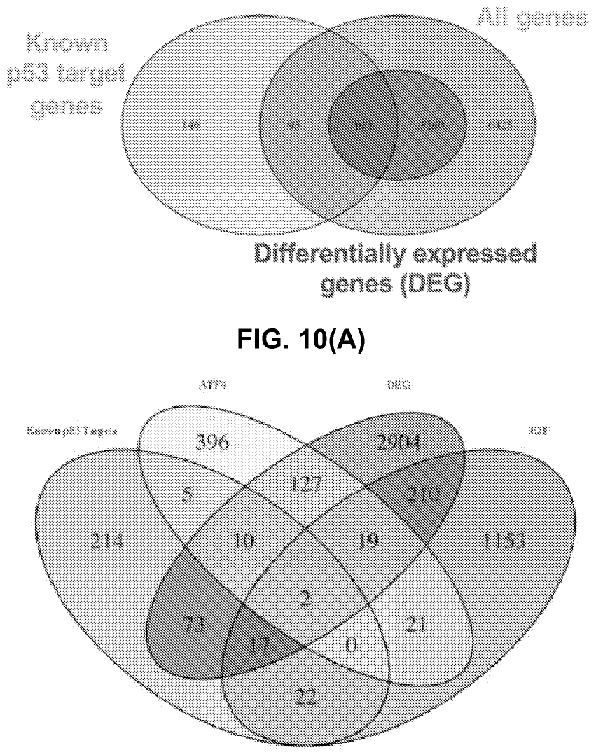

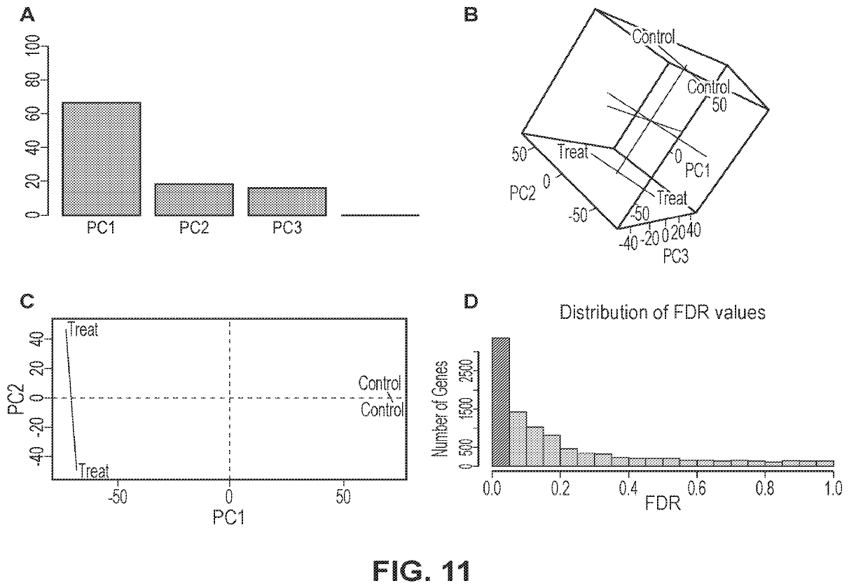

CB002 analog #4 treatment of human tumor cells enriches for cell cycle genes in addition to genes involved in the p53-pathway including apoptosis, indicating p53-pathway functional restoration. To understand how the CB002-analog molecules restore the p53-pathway, The inventors performed a transcriptomic and proteomic analysis in SW480 cells treated with analog #4. To verify the quality of these data, the principal component (PC) plot was obtained. Principal component plots show that the factor with most variability within the samples was the difference between control and treatment. See FIG. 11(A)-(C). Significant differentially expressed genes were defined by a false discovery rate (FDR)<0.05, and 3,362 genes met these criteria. FIG. 11(D). The inventors then identified the differentially expressed genes involved in the p53 pathway. A comprehensive known p53 target gene set used for comparison were the genes previously shown to be directly regulated by p53 through chromatin immunoprecipitation assays (CHIP) assays and genes that were protein-coding genes in at least 3 of the 17 genome wide data sets. See TABLE S3 from Fischer, Oncogene, 36(28), 3943-3956 (2017). Of the 343 genes in the known p53 target gene set, 334 genes were tested in the microarray, but only 197 genes met the low expression cutoff. From the 197 genes that met the low expression criteria, 102 genes were differentially expressed. See FIG. 10(A). Gene ontology (GO) analysis of the 102 differentially expressed genes showed these genes are highly enriched in regulating programmed cell death. See TABLE 3. A gene expression heatmap of these genes shows that most the genes are upregulated by analog #4 treatment of tumor cells. The inventors then performed a transcription factor analysis of all 3,362 differentially expressed genes. Transcription factor analysis defined by direct binding of predictive binding motifs revealed E2F transcription factors as having the highest normalized enrichment score (NES). See TABLE 2. Because the transcription factor ATF4 was important for Noxa induction, the inventors compared a known ATF4 gene set (see TABLE S3 from Wang et al., Mol. Cancer Ther., 14(4), 877-88 (2015) and an E2F gene set (see TABLE S1 from Ren et al., Genes Dev, 16(2), 245-56 (2002) with the known p53 gene set and the differentially expressed genes in analog #4 treatment. See FIG. 10(B). The resulting Venn diagram of this comparison shows that both ATF4 and E2F targets genes are not unique to these transcription factors and also share common targets with p53 (-5%). Analyzing the ratio of differentially expressed genes to the transcription factor gene set did not show an obvious gene enrichment regulation of one transcription factor. See TABLE 4. Despite p53 not being the top predictive transcription factor in this analysis, ingenuity pathway analysis (IPA) determined p53 to be activated as an upstream regulator with a z-score value of 3.3 and p-value of 2.9×10⁻³⁴. A Kyoto Encyclopedia of Genes and Genomes (KEGG) analysis for the p53-pathway signaling was obtained with an adjusted p value (adjp) equal to 1.18×10⁻¹ that despite not reflecting a significant enrichment of the p53 pathway, it shows thirty-one differentially expressed genes from the fifty-two genes tested and present in the KEGG analysis. This accounts for 60% of differentially expressed genes in the KEGG p53-pathway analysis. Consistent with the GO terms results, p53 target genes involved in apoptosis such as Noxa, Puma, and DR5 were upregulated by the analog #4 treatment. Taken together, these data shows that although a large set of genes differentially expressed are not predicted to be directly regulated through direct p53 binding, a subset of these are enriched in the p53-pathway, indicative of p53-pathway restoration.

TABLE 3 shows the enriched biological process GO terms in the 102 differentially expressed genes in CB002 analog #4 treated cells. Gene ontology (GO) analysis for the 102 differentially expressed genes that are also known p53 target genes. GO term analysis was done using the R package “goseq” and those genes enriched in particular biological process are described along with their adjp value. Top 25 enriched GO terms are listed.

TABLE 3
The enriched biological process GO terms

| GO term ID | Name | adjp |
|---|---|---|
| GO: 0008219 | cell death | 4.447579E−08 |
| GO: 0010941 | regulation of cell death | 4.447579E−08 |
| GO: 0012501 | programmed cell death | 5.409503E−08 |
| GO: 0006915 | apoptotic process | 5.466988E−08 |
| GO: 0043067 | regulation of programmed cell death | 2.902278E−07 |
| GO: 0097193 | intrinsic apoptotic signaling pathway | 4.205507E−07 |
| GO: 0097190 | apoptotic signaling pathway | 4.949351E−07 |
| GO: 0042981 | regulation of apoptotic process | 6.67383E−07 |
| GO: 0072331 | signal transduction by p 53 class mediator | 7.248333E−07 |
| GO: 0050896 | response to stimulus | 7.321667E−07 |
| GO: 0007154 | cell communication | 1.104964E−06 |
| GO: 0051716 | cellular response to stimulus | 1.532609E−06 |
| GO: 0023052 | signaling | 2.587082E−06 |
| GO: 0007165 | signal transduction | 2.587082E−06 |
| GO: 0009966 | regulation of signal transduction | 5.993752E−06 |
| GO: 0072332 | Intrinsic apoptotic signaling pathway | 1.671799E−05 |
| | by p 53 class | |
| GO: 0048583 | regulation of response to stimulus | 3.853858E−05 |
| GO: 2001233 | regulation of apoptotic signaling pathway | 3.893307E−05 |
| GO: 0010646 | regulation of cell communication | 4.168569E−05 |
| GO: 0007166 | cell surface receptor signaling pathway | 4.430113E−05 |
| GO: 0023051 | regulation of signaling | 5.136280E−05 |
| GO: 0010942 | positive regulation of cell death | 8.303541E−05 |
| GO: 0043065 | positive regulation of apoptotic process | 1.220935E−04 |
| GO: 0048584 | positive regulation of response to stimulus | 1.220935e−04 |
| GO: 0009968 | negative regulation of signal transduction | 1.220935e−04 |

TABLE 4 shows the contribution of transcription factors P53, ATF4, and E2F to differentially expressed genes in CB002 analog #4 treated cells. The total number of differentially expressed genes that overlapped with known genes of each transcription factor was calculated. This total is reflected in the “number of genes in DEG” column. Using this number, The inventors then calculated the ratio of differentially expressed genes divided by the total of genes in the transcription factor data set.

TABLE 4
Contribution of transcription factors P53, ATF4, and E2F to
differentially expressed genes in CB002 analog #4 treated cells

| Transcription | Number of | | Number | |
|---|---|---|---|---|
| factor | Genes in DEG | | in data set | Ratio |
| P53 | 73 + 10 + 2 + 17 = | 102 | 343 | 0.3 |
| ATF4 | 127 + 10 + 19 + 2 = | 158 | 559 | 0.28 |
| E2F | 17 + 2 + 19 + 210 = | 248 | 1,444 | 0.17 |

The inventors determined the enriched pathways in the whole set of differentially expressed genes (3,362) by performing a KEGG analysis. The top four enriched pathways obtained from the KEGG analysis included cell cycle, DNA repair, mismatch repair (MMR), and nucleotide excision repair (NER). The adjusted p value (adjp) for each KEGG pathway was 2.27×10⁻⁶, 2.27×10⁻⁶, 5.05×10⁻³, 2.18×10⁻², respectively. The adjp values show a significant enrichment score of each pathway. Gene ontology (GO) terms in biological processes also reflected enrichment of genes that participate in cell cycle regulation. TABLE 5. The KEGG analysis and GO ontology both reflected the downregulation of genes involved in the G1/S -phase of the cell cycle in CB002 analog treated cells. E2F is responsible for the induction of genes in DNA initiation and replication such as MCM complexes and origin replication complexes (ORC). Bracken et al., Trends Biochem. Sci., 29(8), 409-17 (2004). The transcriptomic analysis shows downregulation of these genes and the inhibition of E2F transcriptional activity. Downregulation of Cyclin E and Cyclin A genes further confirmed the delay of cells to S-phase. GADD45, a p53-target gene that can induce cell cycle arrest was upregulated. Further study is necessary to validate the direct implication of E2F's and p53 target genes in the perturbation of the delay in S-phase. Nonetheless, these data shows that the identified family of small molecules represent a unique mechanism of action that involves S-phase delay perturbation and p53-pathway restoration.

TABLE 5. Enriched biological process GO terms in the 3,362 differentially expressed genes. Gene ontology (GO) analysis for all differentially expressed genes by analog #4 treatment. GO term analysis was done using the R package “goseq” and those genes enriched in particular biological process are described along with their adjp value. The top 20 enriched GO terms are listed.

TABLE 5
Enriched biological process GO terms in differentially expressed genes

| GO term ID | Name | adjp |
|---|---|---|
| GO: 0022402 | cell cycle process | 7.751840E−16 |
| GO: 0000278 | mitotic cell cycle | 7.751840E−16 |
| GO: 0007049 | cell cycle | 2.753525E−15 |
| GO: 1903047 | mitotic cell cycle process | 2.753525E−15 |
| GO: 0044770 | cell cycle phase transition | 3.654863E−13 |
| GO: 0006260 | DNA replication | 5.207792E−13 |
| GO: 0044772 | mitotic cell cycle phase transition | 1.192425E−11 |
| GO: 0006261 | DNA-dependent DNA replication | 2.919748E−11 |
| GO: 0007059 | chromosome segregation | 3.095763E−11 |
| GO: 0044786 | cell cycle DNA replication | 3.986797E−11 |
| GO: 0051301 | cell division | 1.520253E−10 |
| GO: 0000280 | nuclear division | 1.693587E−09 |
| GO: 0098813 | nuclear chromosome segregation | 1.985723E−09 |
| GO: 0033260 | nuclear DNA replication | 2.161431E−09 |
| GO: 0000819 | sister chromatid segregation | 1.299694E−08 |
| GO: 0044843 | cell cycle G1/S phase transition | 5.548849E−08 |
| GO: 0071103 | DNA conformation change | 6.704071E−08 |
| GO: 0048285 | organelle fission | 7.309501E−08 |
| GO: 0051726 | regulation of cell cycle | 9.496568E−08 |
| GO: 0000070 | mitotic sister chromatid segregation | 1.696716E−07 |

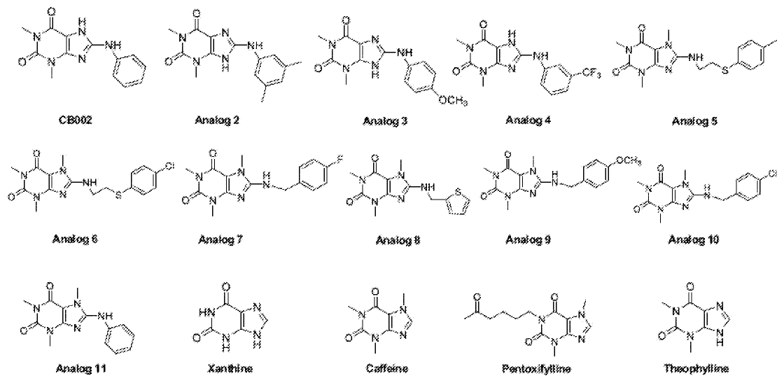

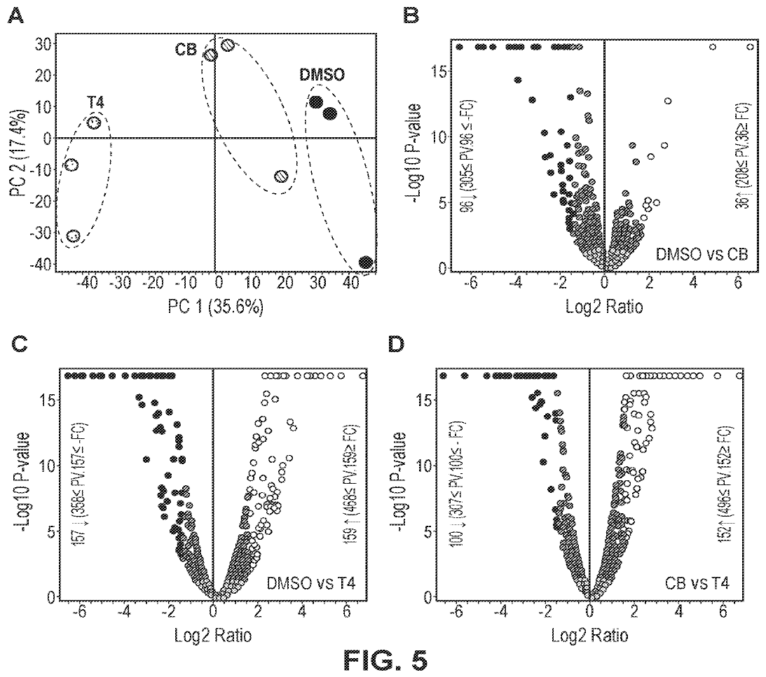

To show that the stimulation of the p53 pathway at the transcriptional level was restoring the p53 pathway at the protein level, a comparative label-free quantitative proteomic analysis of SW480 colon cancer cells in response to DMSO and analog #4 (T4) treated for twenty-four hours was performed. FIG. 5A) shows close clustering of protein abundance of each replicate under the same group, and variability among the treatments. Volcano plots of fold-change versus q-value of the total of 3,743 proteins quantified from SW480 cells in response to DMSO, CB002 (CB) and analog #4 (T4) treatments show differentially expressed proteins determined as significant (p<0.05) up and down. See FIG. 5 (B)-(D). At the protein level, pathway analysis did not reflect an enrichment in p53 targets. See FIG. 2(B). Consistent with the microarray data, the proteomic pathway analysis of the differentially abundant proteins shows downregulation of proteins involved in cell cycle regulation. See FIG. 2(B). CDK4, CKS1B, ERCC6L, MAPK3, and MAX are decreased in analog #4 treatment than in CB002.

As the CB002-analog molecules were discovered as p53 pathway restoring compounds, The inventors compared the proteomic data, with the known p53 target gene set used in the transcriptomic analysis (see TABLE S3 from Fischer, Oncogene, 36(28), 3943-3956 (2017) with a p53-proteomic database (see Tian et al., bioRxiv (2020)). This proteomic database was derived from a comparison of HCT116 vs HCT116 p53^{-/-} cells treated with 5-Fluorouracil (5-FU). These results show that of all significantly upregulated expressed proteins, only 4 overlapped with the known p53 targets and six proteins with p53-proteomics. See FIG. 7(A). Eleven proteins were downregulated by analog #4 treatment overlapping with the in-house proteomic database and none with the known p53 target data set. See FIG. 7(B). No upregulated or downregulated proteins were found to overlap in all three data sets: analog #4 treatment and both reference databases. See FIG. 7(A)-(B).

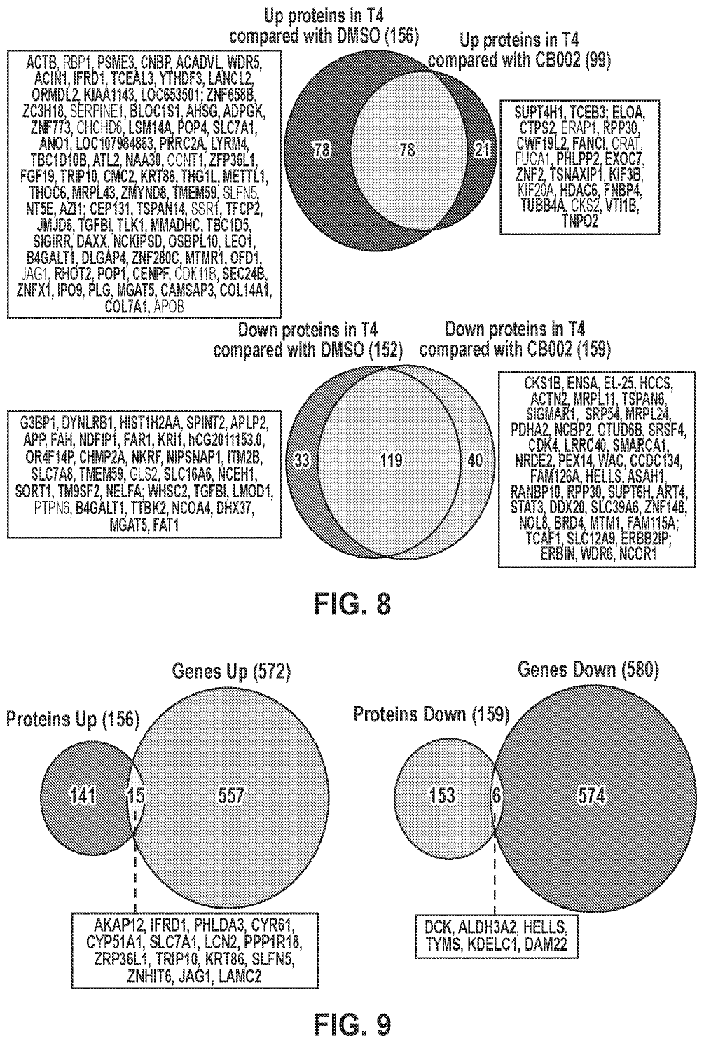

These results show that within the proteins tested in the proteomic analysis, those expressed by analog #4 treatment and involved in the p53 pathway were minimal under the performed experimental conditions. Additional proteins validated by western blot such as Noxa and DR5 were not detected in the proteomic analysis indicating that the proteomic analysis is preliminary. The inventors observed differences at the level of protein expression between parental compound CB002 and its analog #4 both downregulated and to a lesser extent, upregulated proteins. See FIG. 8. These differences show that these small molecules can have different effects in tumor cells albeit they have >50% homology in their proteomic composition.

Discussion. CB002-analog #4 is 20-30 times more potent than CB002. Like its CB002 parental compound, CB002-analog #4 restores the p53-pathway and induces apoptosis independently of p73. The twelve p53 pathway restoring structural analogs of CB002 tested were similar because they resemble the structure of a xanthine. Transcriptional analysis identified 110 genes involved in the p53-pathway. Upstream prediction analysis showed p53 as one of the main upstream transcription regulators with a P-value of 2.9×10⁻³⁴ and 3.3 z-score. See TABLE 6.

TABLE 6
Microarray data upstream prediction analysis for CB002-
analog #4 microarray 110 genes are part of the p53 pathway

| Upstream | Molecule | Predicted | Activation | p-value of |
|---|---|---|---|---|
| Regulator | Type | Activation State | z-score | overlap |
| NUPR1 | transcription | Activated | 8.966 | 3.53E−59 |
| | regulator | | | |
| TP53 | transcription | Activated | 3.3 | 2.9E−34 |
| | regulator | | | |
| E2F4 | transcription | | 0.152 | 1.15E−30 |
| | regulator | | | |
| CCND1 | transcription | Inhibited | −2.968 | 3.16E−26 |
| | regulator | | | |
| ATF4 | transcription | Activated | 4.93 | 2.85E−23 |
| | regulator | | | |
| E2F1 | transcription | Inhibited | −4.826 | 2.9E−21 |
| | regulator | | | |
| RB1 | transcription | Activated | 3.714 | 8.81E−20 |
| | regulator | | | |
| E2F3 | transcription | Inhibited | −2.44 | 6.33E−17 |
| | regulator | | | |

These data further validate this anticancer class of small molecules as being p53-restoring drugs. Microarray analysis identified 150 genes involved in cell cycle regulation, DNA synthesis, and repair that are significantly decreased compared to dimethyl sulfoxide control. These genes include minichromosome maintenance proteins (MCM's), Cyclin E, CDK's, E2F's, and Cdc2. The proteomic analysis also confirmed an increase in proteins regulated by p53 and a decrease in proteins involved in cell cycle regulation. Thus, the transcriptomic and proteomic analyses coincide in that CB002-analog #4 reduces critical regulators of the cell cycle.

The inventors determined the effects of the CB002-analogs on the cell cycle. The data show that the p53-restoring CB002-analog compounds, unlike known xanthines such as caffeine, pentoxifylline, and theophylline, restore the p53 and do not deregulate the G2-checkpoint. Treatment with these small molecule CB002-analogs results in the activation of an S-phase DNA damage response pathway characterized by the increase in p-ATR(Thr1989). This treatment should lead to a delay of cells in the S-phase. This S-phase perturbation should contribute to cancer cell death.

The observed S-phase perturbation can lead to new therapeutic regimens such as synthetic lethality in BRCA-deficient cells and combination with PARP inhibitors.

The inventors previously reported that pro-apoptotic protein Noxa functions in CB002-mediated cell death. Hernandez Borrero et al., Cell Cycle, 17(5), 557-567 (2018). CB002 analogs induce Noxa expression across different colorectal cancer cell lines in vitro. The inventors show Noxa functions in vivo as CB002-analog #4 treatment of SW480 shNoxa tumors does not reduce tumor volume compared to vehicle control. The inventors have evidence indicating that ATF3/4 μlays a role in regulating Noxa as knockdown of ATF3/4 results in decreased Noxa protein expression. The transcriptomic data shows activation of the integrated stress response as shown by the increase of genes involved in the unfolded protein response, tRNA aminoacylation, and increase of ATF3/4 protein expression by Western blot.

The inventors show that CB002-analog #4 induces apoptosis in colorectal cancer patient-derived organoid cells. CB002-analog #4 is safe both in vitro and in vivo, as shown by a lack of increase in the Sub-G1 population in normal human fibroblasts and healthy NSG mice body weight throughout treatment. The observed decrease in tumor volume by CB002-analog #4 depends on Noxa. Because Noxa rarely is mutated in human cancer, its induction by the CB002-analogs offers a feasible therapeutic advantage leading to tumor cell death. Noxa expression can be a pharmacodynamic biomarker to predict therapeutic response. These data show that CB002 and its analogs represent a anti-tumor agent class that provides a unique therapeutic strategy that can be clinically translated.

Defining the p53-pathway restoration mechanism of action. The inventors performed a transcriptomic and proteomic analysis. The microarray data show a significant increase in gene expression of genes regulated by p53, including PMAIP1gene (Noxa), and a decrease in 150 genes involved in cell cycle regulation at twelve hours. See FIG. 2. Ingenuity Pathway upstream prediction analysis shows p53 as an upstream regulator with a z-score value of 3.3. See TABLE 6. A comparative label-free quantitative proteomic analysis of SW480 colon cancer cells in response to dimethyl sulfoxide and CB002-analog #4 (T4) treated for twenty-four hours was performed. FIG. 5(A) shows close data clustering of protein abundance of each replicate under the same group, with variability among the treatments. The volcano-plots of fold-change versus q-value of the total of 3743 proteins quantified from SW480 cells in response to dimethyl sulfoxide, CB002 (CB), and T4 treatments show differentially expressed proteins determined as significant (p<0.05) up and down. See FIG. 5(B)-(D). Reactome pathway analysis also shows a significant decrease in proteins involved in cell cycle regulation. CDK4, CKS1B, ERCC6L, MAPK3, and MAX are decreased in CB002-analog #4 treatment.

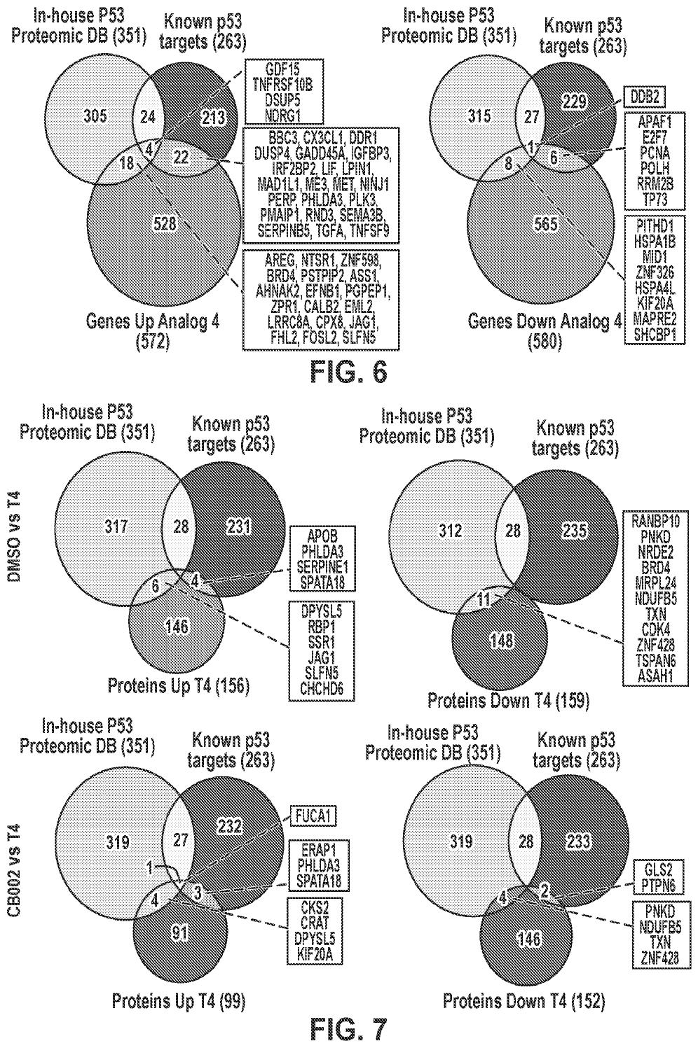

Because the CB002-analog molecules were discovered as p53 restoring compounds, the inventors compared the transcriptomic data with a known p53 target gene database (Fischer, Oncogene, 36(28), 3943-3956 (Jul. 13, 2017)) and the in-house p53-proteomic database. See FIG. 6-FIG. 9. the in-house proteomic database was derived from a comparison of HCT116 vs. HCT116 p53^{-/-} cells treated with 5-fluorouracil (5-FU). These results show that eighteen p53 targets in the in-house p53-proteomics database and twenty-two known p53 targets are common between CB002-analog #4 upregulated genes. All three data sets have four upregulated genes in common. Down-regulated genes by CB002-analog #4 include eight genes in common with the in-house proteomic database and eight in the known p53 target dataset. All three sets had DDB2 in common. Down-regulated genes by CB002-analog #4 include DNA synthesis, repair, and cell cycle genes that are p53 targets, including DDB2, PCNA, POLH, and E2F7.

A p53 reference database was compared to the proteomic upregulated and downregulated genes differentially expressed by CB002-analog #4. There were less commonly expressed proteins between the datasets and CB002-analog #4. Six proteins were upregulated between the in-house proteomic database. Four proteins were upregulated in the known p53 target gene database. Eleven genes were downregulated by CB002-analog #4 treatment and the in-house proteomic database. No genes were found in common to be upregulated or downregulated by CB002-analog #4 treatment and both reference databases.

CB002 and analogs perturb S-Phase but not G2-checkpoint, unlike other known xanthines. Caffeine is a G2-checkpoint deregulator through inhibition of ATM/ATR. Thus, a combination of chemotherapy agents with caffeine results in enhanced cancer cell cytotoxicity. This combination was not pursued due to caffeine's lack of achievable required concentrations in plasma.

The inventors investigated whether CB002 and its analogs can deregulate the G2-checkpoint, like caffeine, pentoxifylline, and theophylline. The inventors synchronized SW480 colon cancer cells using double thymidine block, release, and treatment with CB002 analog compound alone or combined with etoposide and probed for key G2/M-phase cell cycle markers. The inventors observed that etoposide treatment enhances protein expression of pcdc2^{(Tyr15)} and pcdc25c^{(Ser16)}, indicating cell cycle arrest due to DNA damage. The combination of etoposide with caffeine resulted in G2-deregulation, as shown by decreased expression of pcdc2^{(Tyr15)} and pcdc25c^{(Ser16)}.

The combination of etoposide with CB002 or CB002-analog #4 showed a decrease in expression of pcdc2^{(Tyr15)} and pcdc25c^{(Ser16)}. But CB002 or CB002-analog #4 does not increase M-phase marker pH3^{(Ser10)} as expected for a G2-deregulator like caffeine. These data show that CB002 and CB002-analog #4 either do not deregulate the G2-checkpoint or that these compounds delay cells going into M-phase.

CB002-analogs increase p-Cdc25c and p-Cdc2 combined with etoposide indicating cell cycle arrest. The increase in Cyclin E and Cyclin A combined with etoposide shows that these analogs increase S-phase markers and do not deregulate G2-markers.

The inventors performed a similar set of time-course assays after cell synchronization release to further elucidate the cell cycle effects of CB002-analog #4. Cell cycle markers pcdc2^{(Tyr15)} and pcdc25c^{(Ser16)} expression decreased in CB002-analog #4 compared to DMSO and etoposide and their expression over time increased at twelve hours indicative of a delay of cells in the G2 cell cycle phase. To further elucidate the effect in S-phase, we evaluated Cyclin A and p-RPA-RPA2^{(S8)}, the latter as a marker of single stranded DNA and replication stress possibly caused by stalled or collapsed replication forks. Cyclin A expression did not decrease over time in CB002-analog #4 treated cells as compared to DMSO and etoposide indicating that cells were delayed in S-phase. p-RPA-RPA2^{(S8)} expression upon CB002-analog #4 treatment was increased compared to DMSO indicating replication stress. The p53 target p21 was also found to increase in CB002-analog #4 treated cells indicating cell cycle arrest. Taken together, these analogs deregulate an S-phase checkpoint and not a G2 checkpoint.

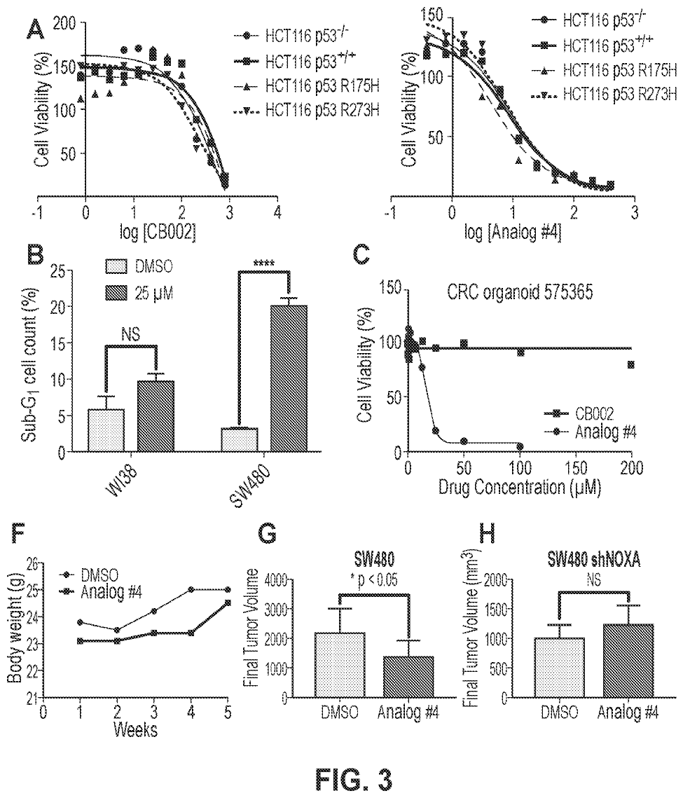

To investigate the effects of these CB002-analogs on the cell cycle, the inventors probed for S-phase specific markers and performed PI analysis upon release of synchronized cells for a time-course of 0-48 hours. CB002 and its structural analogs, unlike caffeine, increase S-phase marker p-RPA-RPA2^{(S8)}, indicating these compounds perturb S-phase. Propidium iodide analysis further confirms that the combination of caffeine deregulates the G2-checkpoint and that CB002-analogs #4 and #10 treatment results in S-phase accumulation, particularly observed at eight hours following release from synchronization. Propidium iodide and BrdU co-staining confirm that CB002-analog #4 increases by 30% cells in S-phase at twelve hours compared to dimethyl sulfoxide vehicle control, and no significant differences are observed in G2-phase cells between etoposide and CB002-analog #4 at twenty-four hours. S-phase delays with CB002 and CB002-analog #10 occur at six-eight hours of treatment, particularly a 2-fold difference combined with etoposide. The caffeine-treated S-phase population is comparable to the dimethyl sulfoxide vehicle control at all time points, indicating that caffeine does not perturb the S-phase. Caffeine decreases the G2-population by 2-fold to 3-fold at twenty-four hours combined with etoposide compared to etoposide alone. No other treatment tested decreases the G2 population when combined with etoposide. See FIG. 3. Haploid cell gating shows the haploid BrdU-positive cells in FIG. 3.

CB002-analog #4 is a potent anti-tumor agent in vitro and in vivo. The inventors focused on lead CB002-analog #4 and investigated its therapeutic index in vitro and in vivo. The inventors treated an isogenic HCT116 cell line panel with 100 μM CB002 or 25 μM CB002-analog #4 and established IC₅₀ values by the Cell-Titer glow cytotoxicity assay. Across this panel, CB002-analog #4 has a 20-fold to 30-fold range in IC₅₀ values, independently of the HCT116 p53-status. The results show that the restoration of the p53-pathway by CB002 or analog #4 is p53-independent.

SW480 cells treated with CB002-analog #4 result in a significant increase of Sub-G1 content compared to vehicle control, whereas treatment with CB002-analog #4 of normal human WI38 lung fibroblast cells does not increase sub-G1 indicating that CB002-analog #4 is safe to normal cells in vitro. CB002-analog #4 is more cytotoxic in vitro than CB002, as shown by clonogenic assay on SW480 cells.

The inventors further investigated the anticancer cytotoxicity potential of CB002-analog #4. The inventors treated a colorectal cancer patient-derived organoid with CB002-analog #4. They performed cellular cytotoxicity analysis in vitro and immunofluorescence staining of ethidium homodimer, calcein, caspase-3, and Ki-67 to distinguish between dead, live, apoptotic, and proliferating cells, respectively. CB002-analog #4 enhances cytotoxicity compared to the CB002 parent compound in the tested colorectal cancer patient-derived organoid as shown by the cell viability response curve. The immunofluorescence assay staining for ethidium homodimer and calcein shows an increase of ethidium homodimer staining of CB002 and CB002-analog #4 to a more significant extent as compared to vehicle control indicating an enhanced killing of cells. Calcein staining shows that organoids treated with CB002-analog #4 are smaller in size, meaning that CB002-analog #4 decreases the growth of the patient-derived organoid. Cleaved caspase-3 staining shows that both CB002 and CB002-analog #4 treatment at IC₅₀ doses increases apoptotic cells. CB002-analog #4 treatment also results in an inverse relationship with Ki-67 staining about drug concentration, indicating that CB002-analog #4 decreases the population of proliferating cells.

The inventors investigated CB002-analog #4 in vivo for anti-tumor efficacy and toxicity in NSG mice. Mice were xenografted with human SW480 colorectal cancer cells treated with CB002-analog #4 at 50 mg/kg by oral gavage three times per week. These data show that CB002-analog #4 is well-tolerated, as shown by the mouse body weights during the duration of the assay. At 5-weeks of treatment, CB002-analog #4 treated tumors have a lower tumor volume than vehicle control.

Mice were xenografted with SW480 cells containing a stable knockdown of Noxa. Mice xenografted with SW480 shNoxa cells did not differ in tumor volume after CB002-analog #4 treatment compared to vehicle control-treated tumors indicating that Noxa is important for reduced tumor volume in vivo.

### LIST OF ADDITIONAL EMBODIMENTS

Specific compositions and methods of making and using small molecule CB002 and analogs were described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as those skilled in the medical chemical art will recognize. When the specification or claims recite method steps or functions in an order, alternative embodiments may perform the functions in a different order or substantially concurrently. Therefore, the inventive subject matter is not to be restricted except in the spirit of the disclosure.

When interpreting the disclosure, all terms should be interpreted in the broadest manner consistent with the context. Unless otherwise defined, all technical and scientific terms used in this specification have the same meaning as commonly understood by a person having ordinary skill in the medical chemical art to which this invention belongs. This invention is not limited to the particular methodology, protocols, reagents, and the like described in this specification and, as such, can vary in practice. The terminology used in this specification is not intended to limit the scope of the invention, which is defined solely by the claims.

Some embodiments of the technology described can be defined according to the following numbered paragraphs:

1. A CB002-analog xanthine derivative, having the following structure:

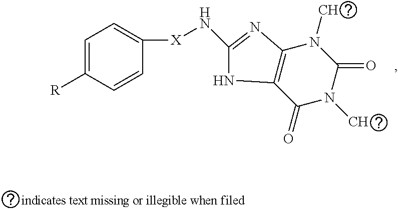

where R=Carbon-based substitutions, aromatic groups, halides, aliphatic chains including attached charged groups, molecule dimers, —Cl, or —CF₃; and X=0, 1, more —CH₂, or other linker groups.

2. The compound of embodiment 1, with the proviso that the class does not include the compound CB002 or the compound CB002-analog #11.

3. The compound of embodiment 1, wherein the compound is CB002-analog #4 (T4).

4. The compound of embodiment 1, wherein the compound is CB002-analog #10.

5. A method of treating subjects with cancer, comprising the step of administering a CB002-analog xanthine derivative to the subject.

6. The method of embodiment 5, wherein the cancer comprises with tumors with mutated p53.

7. The method of embodiment 5, wherein the step of administering a CB002-analog xanthine derivative to the subject further comprises administering to the subject one or more anticancer compounds selected from the group consisting of 5-fluorouracil (5-FU), irinotecan, etoposide, gemcitabine, oxaliplatin, carboplatin, paclitaxel.

8. The method of embodiment 7, wherein the CB002-analog xanthine derivative is CB002-analog #4 (T4).

9. The method of embodiment 7, wherein the CB002-analog xanthine derivative is CB002-analog #10.

10. The method of embodiment 5, wherein the step of administering a CB002-analog xanthine derivative to the subject further comprises administering anticancer radiation treatment to the subject.

11. The method of embodiment 5, wherein the step of administering a CB002-analog xanthine derivative to the subject further comprises administering to the subject one or more anticancer compounds selected from the group of PARP inhibitors.

12. The method of embodiment 11, wherein the CB002-analog xanthine derivative is CB002-analog #4 (T4).

13. The method of embodiment 11, wherein the CB002-analog xanthine derivative is CB002-analog #10.

14. The method of embodiment 5, wherein the step of administering a CB002-analog xanthine derivative to the subject further comprises administering to the subject one or more immune checkpoint therapy compounds selected from the group consisting of an anti-PD-1 antibody, anti-PD-L1 antibody, and anti-CTLA4 antibody.

15. The method of embodiment 14, wherein the CB002-analog xanthine derivative is CB002-analog #4 (T4).

16. The method of embodiment 14, wherein the CB002-analog xanthine derivative is CB002-analog #10.

17. The method of embodiment 5, wherein the step of administering a CB002-analog xanthine derivative to the subject further comprises administering to the subject one or more targeted cancer therapy compounds selected from the group consisting of an anti-ATM antibody, anti-ATR antibody, anti-Chk1 antibody, anti-Chk2 antibody, anti-EGFR antibody, anti-alk antibody, anti-Her2 antibody, anti-NTRK antibody, anti-BRAF antibody, and anti-KRAS antibody.

18. The method of embodiment 17, wherein the CB002-analog xanthine derivative is CB002-analog #4 (T4).

19. The method of embodiment 17, wherein the CB002-analog xanthine derivative is CB002-analog #10.

20. The method of embodiment 5, wherein the cancer comprises DNA repair-deficient tumors that have defective repair of replicating DNA.

21. The method of embodiment 20, wherein the CB002-analog xanthine derivative is CB002-analog #4 (T4).

22. The method of embodiment 20, wherein the CB002-analog xanthine derivative is CB002-analog #10.

23. The method of embodiment 5, wherein the cancer comprises BRCA1-deficient tumors.

24. The method of embodiment 23, wherein the CB002-analog xanthine derivative is CB002-analog #4 (T4).

25. The method of embodiment 23, wherein the CB002-analog xanthine derivative is CB002-analog #10.

### REFERENCES

A person having ordinary skill in the medical chemical art can use the following patents, patent applications, and scientific references as guidance to predictable results when making and using the invention.

### Patent Literature

U.S. Pat. Publ. 2019/0225613 A1 (El-Deiry et al.) Compounds for repressing cancer cell growth described a different approach. The '613 published patent application described the screening for small molecules that restore the p53-pathway instead of direct targeting of p53. The '613 application described compounds, compositions, and methods for cancer treatment by restoring the p53 pathway signaling to repress cancer cell growth. The '613 application started to characterize a new class of small molecules that restore the p53.

### Non-Patent Literature

Abraham, Cell cycle checkpoint signaling through the ATM and ATR kinases. Genes & Dev., 15, 2177-2196 (2001).

Ahsan et al., Highly reproducible improved label-free quantitative analysis of cellular phosphoproteome by optimization of LC-MS/MS gradient and analytical column construction. J. Proteomics, 165, 69-74 (2017).

Bracken et al., E2F target genes: Unraveling the biology. Trends Biochem. Sci., 29(8), 409-17 (2004).

Brown & Baltimore, Essential and dispensable roles of ATR in cell cycle arrest and genome maintenance. Genes Dev., 17(5), 615-628 (Mar. 1, 2003).

Carlsson et al., J. Med. Chem., 53, 3748-3755 (2010).

Chehab et al., Chk2/hCds1 functions as a DNA damage checkpoint in G(1) by stabilizing p53. Genes Dev., 14(3), 278-88 (2000).

Dittmer et al., Gain of function mutations in p53. Nature Genet., 4(1), 42-6 (1993).

Duffy et al., European Journal of Cancer 83 (2017) 258-265 (2017).

Engeland, Cell cycle arrest through indirect transcriptional repression by p53: I have a DREAM. Cell Death Differ., 25(1), 114-132 (2018).

Fischer et al., Integration of TP53, DREAM, MMB-FOXM1 and RB-E2F target gene analyses identifies cell cycle gene regulatory networks. Nucleic Acids Res, 44(13), 6070-86 (2016).

Fischer, Census and evaluation of p53 target genes. Oncogene, 36(28), 3943-3956 (Jul. 13, 2017).

Fracasso et al., A Phase 1 study of UCN-01 in combination with irinotecan in patients with resistant solid tumor malignancies. Cancer Chemother. Pharmacol., 67(6), 1225-37 (2011).

Hernandez Borrero et al., CB002, a novel p53 tumor suppressor pathway-restoring small molecule induces tumor cell death through the pro-apoptotic protein NOXA. Cell Cycle, 17(5), 557-567 (2018).

Huang et al., Discovery of a novel series of CHK1 Kinase inhibitors with a distinctive hinge binding mode. ACS Med Chem Lett., 3(2), 123-128 (February 2012).

Lang et al., Gain of function of a p53 hot spot mutation in a mouse model of Li-Fraumeni syndrome. Cell, 119(6), 861-72 (2004).

Lelo et al., Assessment of caffeine exposure: caffeine content of beverages, caffeine intake, and plasma concentrations of methylxanthines. Clin. Pharmacol. Ther., 39(1), 54-9 (1986).

Muller & Vousden, Mutant p53 in cancer: new functions and therapeutic opportunities. Cancer Cell, 25(3), 304-17 (2014).

Olivier, Hollstein, & Hainaut, TP53 mutations in human cancers: origins, consequences, and clinical use. Cold Spring Harb. Perspect. Biol., 2(1), a001008 (2010).

Ren et al., E2F integrates cell cycle progression with DNA repair, replication, and G(2)/M checkpoints. Genes Dev, 16(2), 245-56 (2002).

Richardson et al., Small-molecule CB002 restores p53 pathway signaling and represses colorectal cancer cell growth, Cell Cycle, 16(18), 1719-1725 (2017).

Riley et al., Transcriptional control of human p53-regulated genes. Nature Rev. Mol. Cell. Biol., 9(5), 402-12 (2008).

Rogers et al., CHK1 inhibition is synthetically lethal with loss of B-family DNA polymerase function in human lung and colorectal cancer cells. Cancer Res. (Mar. 11, 2020).

Russell et al., Abrogation of the G2 Checkpoint Results in Differential Radiosensitization of G1 Checkpoint-deficient and G1 Checkpoint-competent Cells, Cancer Drug Design and Discovery, Stephen Neidle, ed. (Elsevier/Academic Press, 2008), pages 427-431.

Russell et al., Abrogation of the G2 checkpoint results in differential radiosensitization of G1 checkpoint-deficient and G1 checkpoint-competent cells. Cancer Res., 55(8), 1639-42 (1995).

Sarkaria et al., Inhibition of ATM and ATR kinase activities by the radiosensitizing agent, caffeine. Cancer Res., 59(17), 4375-82 (Sep. 1, 1999).

Sarkariaet al., Inhibition of ATM and ATR kinase activities by the radiosensitizing agent, caffeine. Cancer Res., 59(17), 4375-82 (1999).

Smith et al., Chapter 3—The ATM-Chk2 and ATR-Chk1 pathways in DNA damage signaling and cancer. Adv. Cancer Res., 108, 73-112 (2010).

Tian et al., p53-independent partial restoration of the p53 pathway in tumors with mutated p53 through ATF4 transcriptional modulation by ERK1/2 and CDK9. Neoplasia, 23(3), 304-325 (2021).

Tian et al., p53-independent restoration of p53 pathway in tumors with mutated p53 through ATF4 transcriptional modulation by ERK1/2 and CDK9. bioRxiv (2020).

Wang et al., ATF4 gene network mediates cellular response to the anticancer PAD Inhibitor YW3-56 in triple-negative breast cancer cells. Mol. Cancer Ther., 14(4), 877-88 (2015).

Wang, Kim, & El-Deiry, Small-molecule modulators of p53 family signaling and antitumor effects in p53-deficient human colon tumor xenografts. Proc. Natl. Acad. Sci. USA, 103(29), 11003-8 (2006).

Wattel et al., p53 mutations are associated with resistance to chemotherapy and short survival in hematologic malignancies. Blood, 84(9), 3148-57 (1994).

Xu et al., Gain of function of mutant p53 by coaggregation with multiple tumor suppressors. Nat Chem Biol, 7(5), 285-95 (2011).

Zhao & Piwnica-Worms, ATR-mediated checkpoint pathways regulate phosphorylation and activation of human Chk1. Mol Cell Biol., 21(13), 4129-39 (July 2001).

Zimmer et al., The Ohio Journal of Science, 63(3), 97-102 (May 1963).

### TEXTBOOKS AND TECHNICAL REFERENCES

Advanced Organic Chemistry, 4th Edition, by Jerry March, (Wiley Interscience).

Current Protocols in Immunology (CPI) (2003). John E. Coligan, ADA M Kruisbeek, David H Margulies, Ethan M Shevach, Warren Strobe, (eds.) John Wiley and Sons, Inc.

Current Protocols in Molecular Biology (CPMB), (2014). Frederick M. Ausubel (ed.), John Wiley and Sons.

Current Protocols in Protein Science (CPPS), (2005). John E. Coligan (ed.), John Wiley and Sons, Inc.

Immunology (2006). Werner Luttmann, published by Elsevier.

Janeway's Immunobiology, (2014). Kenneth Murphy, Allan Mowat, Casey Weaver (eds.), Taylor & Francis Limited.

Laboratory Methods in Enzymology: DNA, (2013). Jon Lorsch (ed.) Elsevier.

Lewin's Genes XI, (2014). published by Jones & Bartlett Publishers.

Molecular Biology and Biotechnology: a Comprehensive Desk Reference, (1995). Robert A. Meyers (ed.), published by VCH Publishers, Inc.

Molecular Cloning: A Laboratory Manual, 4th ed., Michael Richard Green and Joseph Sambrook, (2012). Cold Spring Harbor Laboratory Press, Cold Spring Harbor, N.Y., USA.

The Encyclopedia of Molecular Cell Biology and Molecular Medicine, Robert S. Porter et al. (eds.), published by Blackwell Science Ltd., 1999-2012.

The Merck Manual of Diagnosis and Therapy, 19^{th} edition (Merck Sharp & Dohme Corp., 2018).

Pharmaceutical Sciences 23^{rd} edition (Elsevier, 2020).

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

We claim:

1. A CB002-analog xanthine derivative, having the following structure: 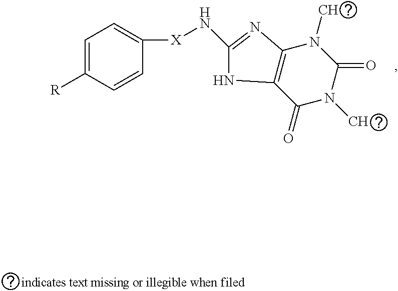
where
R=Carbon-based substitutions, aromatic groups, halides, aliphatic chains including attached charged groups, molecule dimers, —Cl, or —CF₃; and X=0, 1, more —CH₂, or other linker groups.

2. The compound of claim 1, wherein the compound is CB002-analog #4 (T4).

3. The compound of claim 1, wherein the compound is CB002-analog #10.

4. A method of treating subjects with cancer, comprising the step of:
administering a CB002-analog xanthine derivative to the subject.
